(19) United States
(12) Patent Application Publication
(10) Pub. No.: US 2023/0210992 A1
(43) Pub. Date: Jul. 6, 2023

(54) NANOSATELLITE-SUBSTRATE COMPLEX AND METHOD OF REGULATING STEM CELL ADHESION AND DIFFERENTIATION USING THE SAME

(71) Applicant: Korea University Research and Business Foundation, Seoul (KR)

(72) Inventors: Young-Keun KIM, Seoul (KR); Heemin KANG, Seoul (KR); THOMAS MYEONGSEOK KOO, Seoul (KR); Myeong-Soo KIM, Suwon-si (KR); Sung-Gue LEE, Siheung-si (KR); Gun-Hyu BAE, Hwaseong-si, (KR)

(73) Assignee: Korea University Research and Business Foundation, Seoul (KR)

(21) Appl. No.: 17/836,403

(22) Filed: Jun. 9, 2022

(30) Foreign Application Priority Data
Dec. 30, 2021 (KR) 10-2021-0192357

(51) Int. Cl. A61K 41/00; C12N 13/00; C12N 5/0775; A61N 2/00

(52) U.S. Cl. CPC A61K 41/00; C12N 13/00; C12N 5/0662; A61N 2/004

(57) ABSTRACT

The present invention relates to a nanosatellite-substrate complex capable of regulating stem cell adhesion and differentiation, and a method for preparing the same. Moreover, the present invention relates to a method of regulating stem cell adhesion and differentiation by applying a magnetic field to the nanosatellite-substrate complex.

### CROSS-REFERENCE TO RELATED APPLICATIONS

This application claims priority to and the benefit of Korean Patent Application No. 10-2021-0192357 filed in the Korean Intellectual Property Office on Dec. 30, 2021, the entire contents of which are incorporated herein by reference.

### BACKGROUND OF THE INVENTION

### Field of the Invention

The present invention relates to a nanosatellite-substrate complex, a method for preparing the same, and a method of regulating stem cell adhesion and differentiation using the same, and more particularly, to a nanosatellite-substrate complex capable of regulating stem cell adhesion and differentiation and a method of regulating stem cell adhesion and differentiation using the same.

### Description of the Prior Art

Physical screens, which occur in the extracellular matrix (ECM), separate various tissue compartments to help modulate homeostasis and tissue regeneration by controlling biomolecular transport and cellular infiltration. Certain tissues can act as physical screens to modulate tissue repair mechanisms that involve the interactions of diverse cells. However, ECM-mimicking artificial materials that can dispersively and dynamically control bioactive surfaces are rare.

Integrin dynamically forms links with the bioactive-ligand-displaying-ECM, of which the RGD ligands mediate focal adhesion and intracellular mechanotransduction of cells. Remote manipulation of unscreening the ligands by light or magnetic fields can dynamically modulate cell adhesion. Conventionally, light such as ultraviolet (UV), visible, and near infrared (NIR) light has been used for photochemical manipulation of screening and unscreening of the ligands. For example, UV light has been applied to chemically cleave photosensitive polyethylene glycol-based brushes to unscreen ligand-grafted gold nanoparticles for facilitating cell adhesion. Using photoisomers such as azobenzene derivatives, screening and unscreening of the ligands via self-assembled brushes can be manipulated by illuminating UV light and visible light or single wavelengths having the ability to stimulate intracellular mechanotransduction. However, manipulation of screening and unscreening of the ligands by light in vivo has rarely been reported.

In addition, magnetic field can easily penetrate tissues in vivo to enable noninvasive control of physical screens. For example, cell adhesion can be remotely controlled by controlling screening and unscreening of the ligands through modulation of nanoparticles having magnetic properties.

### SUMMARY OF THE INVENTION

An object of the present invention is to provide a nanosatellite-substrate complex which has magnetic properties and is capable of regulating stem cell adhesion and differentiation.

Another object of the present invention is to provide a method for preparing the nanosatellite-substrate complex.

Still another object of the present invention is to provide a method of regulating stem cell adhesion and differentiation using the nanosatellite-substrate complex.

According to one aspect of the present invention, one embodiment of the present invention may include a nanosatellite-substrate complex for regulating stem cell adhesion and differentiation comprising: a substrate; a core-shell-type magnetic particle provided to be spaced apart from at least one side of the substrate; a gold nanoparticle connected to one side of the magnetic particle; a first linker connecting between the substrate and the gold nanoparticle; a second linker connecting between the gold nanoparticle and a ligand; and the ligand connected to the gold nanoparticle via the second linker, wherein the gold nanoparticle is connected to the ligand via the second linker to form a nanoassembly, the magnetic particle is conjugated to the nanoassembly to form a nanosatellite structure, the nanoassembly comprises one or more gold nanoparticles, one or more second linkers connected to at least one of the one or more gold nanoparticles, and the ligand connected to the second linker, and one or more nanoassemblies are comprised in the nanosatellite structure.

The magnetic particle may be composed of: a core composed of iron oxide; and a shell provided to cover the outer surface of the core and comprising silica.

The diameter of the gold nanoparticles may include at least one of a first average diameter, a second average diameter, and a third average diameter, wherein the first average diameter may be 3.5 nm to 10.5 nm, the second average diameter may be 12 nm to 14 nm, and the third average diameter may be 15 nm to 25 nm.

A plurality of the nanoassemblies may be provided adjacent to each other, and the distance between the gold nanoparticles in the nanoassemblies provided adjacent to each other may include at least one of a first distance, a second distance and a third distance, wherein the first distance may be 3 nm to 4 nm, the second distance may be 15 nm to 20 nm, and the third distance may be 18 nm to 22 nm.

The nanoassembly may be provided to completely cover the outer surface of the magnetic particle.

The magnetic particle may have an average diameter of 150 nm to 250 nm, and the magnetic particle may have at least one of an amino group (—HN₂) and a thiol group (—SH) on a surface thereof.

The first linker and the second linker may have a structure of the following Formula 1:

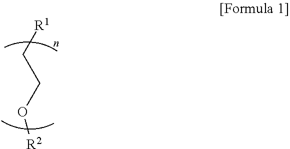

wherein R¹ is one of a thiol group (—SH) and an amine group (—NH₂), R² is one of a carboxyl group (—COOH), an amine group (—NH₂) and a succinimidyl ester group, and n is a number ranging from 113 to 450.

The ligand may be a cyclic RGD ligand.

A surface of the nanosatellite structure, which faces the substrate, may be spaced apart from the substrate with the first linker interposed therebetween, and the first linker may be elastic and the length thereof may be reversibly changed by application of a magnetic field.

The nanosatellite structure may be provided to be spaced apart from one side of the substrate, and the first linker may be compressed by applying a magnetic field to the other side of the substrate, and the nanosatellite structure may move in a direction toward the substrate to facilitate stem cell adhesion and differentiation.

The nanosatellite structure may be provided to be spaced apart from one side of the substrate, and the first linker may be stretched by applying a magnetic field to the upper side of the nanosatellite structure, which is one side of the substrate, and the nanosatellite structure may move in a direction away from the substrate to inhibit stem cell adhesion and differentiation.

The density of the nanosatellite structure provided on the substrate may be 1.0 nanosatellite structure/μm² to 6 nanosatellite structures/μm².

One embodiment of the present invention may include a method for preparing a nanosatellite-substrate complex for regulating stem cell adhesion and differentiation, the method comprising: coating the surface of iron oxide with a silica having an amine group to form magnetic particles; providing gold nanoparticles on the surfaces of the magnetic particles; adding and dispersing the magnetic particles having the gold nanoparticles provided thereon in a solution containing a polymer linker to form a first linker and a second linker; reacting the first linker with a substrate having amine groups formed thereon, so that the first linker is bound to at least a portion of the amine groups formed on the substrate and the magnetic particles having the gold nanoparticles provided thereon are conjugated to the substrate; deactivating amine groups, which remain unbound to the first linker on the substrate, by treatment with a deactivating group; and conjugating a ligand to the second linker.

The diameter of the gold nanoparticles may be any one of a first average diameter, a second average diameter and a third average diameter. The first average diameter of the gold nanoparticles may be 3.5 nm to 10.5 nm, the gold nanoparticles having the first average diameter may be formed by reacting first gold seed particles with amine groups on the surfaces of the magnetic particles to provide the gold seed particles on the magnetic particles, and adding and stirring the magnetic particles having the gold seed particles provided thereon in a gold-containing solution to grow the gold seed particles. Alternatively, the second average diameter of the gold nanoparticles may be 12 nm to 14 nm, the third average diameter thereof may be 15 nm to 25 nm, and the gold nanoparticles having the second or third average diameter may be provided on the magnetic nanoparticles by adding and stirring second gold seed particles in a gold-containing solution to grow the second gold seed particles, thereby forming gold nanoparticles, and reacting the gold nanoparticles, formed by growing the second gold seed particles, with amine groups on the surfaces of the magnetic particles.

The gold-containing solution may include a first solution containing sodium citrate and a second solution containing chloroauric acid, wherein the first solution and the second solution may be sequentially added, and the average diameter of the gold nanoparticles may be controlled by controlling the number of times the first solution and the second solution are added.

One embodiment of the present invention may include a method of regulating stem cell adhesion and differentiation using the nanosatellite-substrate complex, the method comprising regulating stem cell adhesion and differentiation by applying a magnetic field to the nanosatellite-substrate complex having the above-described characteristics.

The magnetic field may be applied from outside the body to remotely control the nanosatellite-substrate complex in the body.

The magnetic field may have a strength of 100 mT to 500 mT.

The nanosatellite structure may be provided to be spaced apart from one side of the substrate, and a plurality of the nanoassemblies may be provided adjacent to each other in the nanosatellite structure. The gold nanoparticles may have an average diameter of 3.5 nm to 10.5 nm, and the distance between the gold nanoparticles in the nanoassemblies provided adjacent to each other may be 3 nm to 4 nm. The first linker may be stretched by applying the magnetic field to the upper side of the nanosatellite structure, which is one side of the substrate, and the nanosatellite structure may move in a direction away from the substrate to inhibit stem cell adhesion and differentiation.

The nanosatellite structure may be provided to be spaced apart from one side of the substrate, and a plurality of the nanoassemblies may be provided adjacent to each other in the nanosatellite structure. The gold nanoparticles may have an average diameter of 15 nm to 25 nm, and the distance between the gold nanoparticles in the nanoassemblies provided adjacent to each other may be 18 nm to 22 nm. The first linker may be compressed by applying the magnetic field to the other side of the substrate, and the nanosatellite structure may move in a direction toward the substrate to facilitate stem cell adhesion and differentiation.

### BRIEF DESCRIPTION OF THE DRAWINGS

The patent or application file contains a least one drawing executed in color. Copies of this patent or patent application publication with color drawing(s) will be provided by the Office upon request and payment of the necessary fee.

FIG. 1 schematically shows a process of regulating stem cell adhesion and differentiation by applying a magnetic field to a nanosatellite-substrate complex according to one embodiment of the present invention.

FIG. 2 schematically shows a process for preparing a nanosatellite-substrate complex.

FIG. 3 shows the results of analyzing the characteristics of magnetic particles.

FIG. 4 shows the results of analyzing the characteristics of gold nanoparticles.

FIGS. 5 to 8 show the results of analyzing nanosatellite-substrate complexes prepared by varying the nanoassembly density.

FIG. 9 shows the results of observing changes after applying a magnetic field to the nanosatellite-substrate complex.

FIG. 10 shows confocal immunofluorescence images and quantification graphs for stem cells after 48 hours of culturing with nanosatellite-substrate complexes obtained by varying the nanoassembly density.

FIG. 11 shows the results of examining whether stem cell adhesion and differentiation occurs on a nanosatellite-substrate complex that does not contain the ligand.

FIG. 12 shows the results of examining whether stem cell adhesion and differentiation occurs on a nanosatellite-substrate complex containing the RAD ligand.

FIG. 13 is a schematic view showing nanosatellite-substrate complexes which comprise gold nanoparticles having different sizes and have different distances between nanoassemblies.

FIG. 14 shows the TEM images, UV-Vis absorbance graphs and diameters of gold nanoparticles.

FIG. 15 shows a process of growing gold nanoparticles after conjugation to magnetic particles and shows the results of observing the characteristics of the gold nanoparticles.

FIG. 16 shows the results of observing the characteristics of each of “S”, “M” and “L” groups.

FIGS. 17 to 19 show the results of measuring the UV-Vis absorbance peaks, hydrodynamic diameters, XRD patterns, and magnetic moments of gold nanoparticles and magnetic particles of “S”, “M” and “L” groups, respectively.

FIG. 20 shows the results of FT-IR measurement performed in each step of conjugating nanoassemblies to magnetic particles.

FIG. 21 shows the results of observation performed after preparing a nanosatellite-substrate complex by conjugating nanoassembly-conjugated magnetic particles to a substrate.

FIG. 22 shows the results of examining whether integrin binds to the nanosatellite-substrate complex.

FIGS. 23 to 25 show the results of experiments conducted on stem cell adhesion and differentiation using nanosatellite-substrate complexes obtained by tuning the diameter of gold nanoparticles and the distance between adjacent gold nanoparticles.

FIG. 26 shows the results of experiments conducted on stem cell adhesion and differentiation using a nanosatellite-substrate complex that does not contain the ligand.

FIG. 27 shows the results of experiments conducted on stem cell adhesion and differentiation using a nanosatellite-substrate complex containing the RAD ligand.

FIGS. 28 and 29 show the results of experiments conducted on the degree of stem cell adhesion and differentiation using nanosatellite-substrate complexes obtained by maintaining the size of gold nanoparticles at 6.5 nm and tuning only the distance between gold nanoparticles.

FIGS. 30 and 31 show the results of experiments conducted on the degree of stem cell adhesion by applying a magnetic field to a nanosatellite-substrate complex.

FIGS. 32 to 34 show the results of comparing the degree of the stem cell adhesion and marker expression caused by applying a magnetic field to nanosatellite-substrate complexes obtained by varying the density of nanoassemblies conjugated to magnetic particles.

FIGS. 35 and 36 show the results of an experiment conducted by culturing for 48 hours in the presence of an inhibitor specific to each protein.

FIGS. 37 and 38 show the results of experiments conducted on stem cell adhesion and differentiation by dividing gold nanoparticles into “S”, “M” and “L” groups based on the diameter of the gold nanoparticles and the distance between adjacent gold nanoparticles and applying a magnetic field to the “L” group.

FIG. 39 shows the results of an experiment conducted on stem cell adhesion and differentiation by applying a magnetic field to a nanosatellite-substrate complex that does not contain the ligand.

FIG. 40 shows the results of an experiment conducted on stem cell adhesion and differentiation by applying a magnetic field to a nanosatellite-substrate complex containing the RAD ligand.

FIGS. 41 to 43 show the results of experiments conducted by in vivo implantation of nanosatellite-substrate complexes prepared by changing the nanoassembly density.

FIGS. 44 and 45 show the results of experiments conducted by in vivo implantation of nanosatellite-substrate complexes obtained by tuning the diameter of gold nanoparticles and the distance between adjacent gold nanoparticles.

### DETAILED DESCRIPTION OF THE INVENTION

The details of other embodiments are included in the detailed description and the accompanying drawings.

The advantages and features of the present invention, and the way of attaining them, will become apparent with reference to the embodiments described below in conjunction with the accompanying drawings. However, the present disclosure is not limited to the embodiments disclosed below and may be embodied in a variety of different forms. Since all numbers, values and/or expressions referring to quantities of components, reaction conditions, etc., used in the present specification, are subject to the various uncertainties of measurement encountered in obtaining such values, unless otherwise indicated, all are to be understood as modified in all instances by the term “about.” Where a numerical range is disclosed herein, such a range is continuous, inclusive of both the minimum and maximum values of the range as well as every value between such minimum and maximum values, unless otherwise indicated. Still further, where such a range refers to integers, every integer between the minimum and maximum values of such a range is included, unless otherwise indicated.

In the present specification, where a range is stated for a parameter, it will be understood that the parameter includes all values within the stated range, inclusive of the stated endpoints of the range. For example, a range of 5 to 10 will be understood to include the values 5, 6, 7, 8, 9, and 10, as well as any sub-range such as 6 to 10, 7 to 10, 6 to 9, and 7 to 9, and also include any value and range between the integers which are reasonable in the context of the range stated, such as 5.5, 6.5, 7.5, 5.5 to 8.5 and 6.5 to 9. For example, a range of “10% to 30%” will be understood to include the values 10%, 11%, 12%, 13%, etc., and all integers up to and including 30%, as well as any sub-range such as 10% to 15%, 12% to 18%, 20% to 30%, etc., and also include any value between the integers which are reasonable in the context of the range stated, such as 10.5%, 15.5%, 25.5%, etc.

In the present specification, the term “nanoassembly” refers to a structure in which a gold nanoparticle and a ligand are connected to each other through a second linker, the term “nanosatellite structure” refers to a structure in which the nanoassembly is conjugated to a magnetic particle, and the term “nanosatellite-substrate complex” refers to a structure in which the nanosatellite structure is conjugated to a substrate via a first linker.

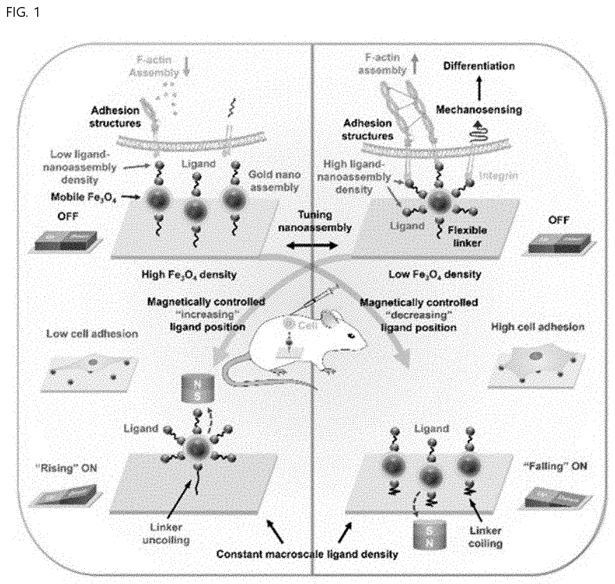

FIG. 1 schematically shows a process of regulating stem cell adhesion and differentiation using a nanosatellite-substrate complex according to one embodiment of the present invention.

According to one embodiment of the present invention, a nanosatellite-substrate complex for regulating stem cell adhesion and differentiation according to the present invention comprises: a substrate; a core-shell-type magnetic particle provided to be spaced apart from at least one side of the substrate; a gold nanoparticle connected to one side of the magnetic particle; a first linker connecting between the substrate and the gold nanoparticle; a second linker connecting between the gold nanoparticle and a ligand; and the ligand connected to the gold nanoparticle via the second linker, wherein the gold nanoparticle, the second linker and the ligand may form a nanoassembly, and the nanoassembly may comprise one or more gold nanoparticles, one or more second linkers connected to at least one of the one or more gold nanoparticles, and a ligand connected to the second linkers, and one or more nanoassemblies may be comprised in the nanosatellite structure.

The substrate may have an amino group (—NH₂) on the surface thereof, and the first linker connected to one side of the magnetic particle may be conjugated thereto. Since the magnetic particle has magnetic properties, movement of the magnetic particle may be induced when a magnetic field is applied to the magnetic particle. Thus, it is possible to regulate stem cell adhesion and differentiation by moving the magnetic particle. The magnetic particle may have a core-shell structure, and the core may be composed of a magnetic material, preferably iron oxide. The shell may be provided to cover the outer surface of the core and may be composed of a shell comprising silica. The surface of the shell has a functional group capable of binding to the gold nanoparticles, and thus the gold nanoparticles may be conjugated to the magnetic particle. The functional group capable of binding to the gold nanoparticles may be, for example, at least one of an amino group (—NH₂) and a thiol group (—SH).

The magnetic particle may have a diameter sufficient to allow the gold nanoparticles to be conjugated thereto. The average diameter of the magnetic particle may be 150 nm to 250 nm.

Gold nanoparticles may be provided on one side of the magnetic particle.

When the functional group on the surface of the magnetic particle is an amino group, the gold nanoparticles may be provided by Au-amino bonding with the amino group on the surface of the magnetic particle. One or more gold nanoparticles may be provided on the surface of the magnetic particle.

The diameter of the gold nanoparticles may include at least one of a first average diameter, a second average diameter, and a third average diameter, wherein the first average diameter may be 3.5 nm to 10.5 nm, the second average diameter may be 12 nm to 14 nm, and the third average diameter may be 15 nm to 25 nm. The diameter of the gold nanoparticles may be a diameter sufficient to allow a ligand to be conjugated thereto.

When the diameter of the gold nanoparticles is the first average diameter, the gold nanoparticles may be formed by conjugating gold seed particles smaller than the first average diameter to the magnetic particle and then growing the gold seed particles on the magnetic particle. In addition, when the diameter of the gold nanoparticles is any one of the second average diameter and the third average diameter, gold nanoparticles having the second average diameter or the third average diameter may be formed and then conjugated to the magnetic particle.

A polymer linker may be conjugated to the gold nanoparticles connected to one side of the magnetic particle. The polymer linker may have a structure of Formula 1 below. The first binding site of the polymer linker may include at least any one of an amino group (—NH₂) and a thiol group (—SH), and may be represented by R¹ in Formula 1. The second binding site of the polymer linker may have at least any one of a carboxyl group (—COOH), an amino group (—NH₂) and a succinimidyl ester group, and may be represented by R² in Formula 1.

In addition, n in Formula 1 may be a number ranging from 113 to 450, preferably from 113 to 225.

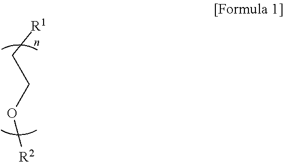

The polymeric linker may include a first linker and a second linker.

The first binding site of the first linker may bind to the gold nanoparticle, and the second binding site thereof may bind to the substrate and connect between the gold nanoparticle and the substrate. The second binding site may be connected by chemical bonding with an amino group on the substrate.

The first binding site of the second linker may bind to the gold nanoparticle, and the second binding site thereof may bind to the ligand and connect between the gold nanoparticle and the ligand. One or more first linkers and one or more second linkers may be conjugated to each of the gold nanoparticles.

The ligand may be a site which is connected to the gold nanoparticle and to which stem cell integrin binds.

The ligand connected to the gold nanoparticle via the second linker may be a cyclic RGD ligand. The RGD may be a ligand having a tripeptide structure consisting of arginine-glycine-aspartame (Arg-Gly-Asp).

The ligand may be a ligand having an amino group (—NH₂).

The ligand may be a ligand having a lysine residue bound thereto.

One or more ligands may be conjugated to each of the gold nanoparticles.

The gold nanoparticle and the ligand may be connected to each other via the second linker to form a nanoassembly. One or more nanoassemblies may be connected to one side of the magnetic particle.

When a plurality of the nanoassemblies are comprised in the nanosatellite structure, the nanoassemblies may be provided adjacent to each other. The distance between the nanoassemblies provided adjacent to each other may be determined by the distance between the gold nanoparticles included in the nanoassemblies. The distance between the gold nanoparticles in the adjacent nanoassemblies may include at least one of a first distance, a second distance, and a third distance. Here, the first distance may be 3 nm to 4 nm, the second distance may be 15 nm to 20 nm, and the third distance may be 18 nm to 22 nm.

In addition, the nanoassemblies may be provided to completely cover the outer surface of the magnetic particle.

The nanoassemblies may be conjugated to the magnetic particle to form a nanosatellite structure.

As the distance between the adjacent gold nanoparticles decreases, the distance between the ligands connected to the gold nanoparticles may decrease, and as the distance between the ligands decreases, stem cells can recognize the gold nanoparticle-conjugated as connected ligands. Thus, as the distance between the adjacent gold nanoparticles decreases, the density of stem cell integrin that bind to the ligands may increase, whereby integrin clustering may occur and stem cell differentiation may be facilitated.

For example, when the adjacent gold nanoparticles have the first distance, stem cells can recognize the ligands in the same way as when the nanoassemblies are provided to completely cover the outer surface of the magnetic particle, and stem cell differentiation may be facilitated.

When the adjacent gold nanoparticles have the first distance, the gold nanoparticles may be formed by a method of growing the gold seed particles on the surface of the magnetic particle to form gold nanoparticles. In this case, the distance between the adjacent gold seed particles may be similar to or larger than the Debye length, but the distance between the adjacent gold nanoparticles formed by growth of the gold seed particles may be similar to or smaller than the Debye length. Accordingly, when the distance between the adjacent gold nanoparticles has the first distance, the ligand conjugated to each of the gold nanoparticles may bind to a cell to form a quasi-connection, and stem cells may recognize the ligands in the same way as when the nanoassemblies are provided to completely cover the outer surface of the magnetic particle. Thus, stem cell adhesion and differentiation may be facilitated.

On the other hand, as the distance between the adjacent gold nanoparticles increases, the distance between the ligands connected to the gold nanoparticles may increase, so that the density of stem cell integrin that binds to the ligands may decrease and stem cell differentiation may be inhibited. For example, when the adjacent gold nanoparticles have the third distance, stem cell differentiation may be inhibited.

The density of the nanosatellite structure provided on the substrate may be kept constant, and the density may be 1.0 nanosatellite structure/μm² to 6.0 nanosatellite structures/μm².

A surface of the nanosatellite structure, which faces the substrate, may be spaced apart from the substrate with the first linker therebetween, and the first linker may be elastic and the length thereof may be reversibly changed by application of a magnetic field.

When the nanosatellite structure is moved by applying a magnetic field to the nanosatellite-substrate complex, stem cell adhesion and differentiation may be regulated. For example, when the first linker is compressed by applying a magnetic field and the nanosatellite structure moves in a direction toward the substrate, the nanosatellite structure may be fastened closely to the substrate, and stem cell adhesion and differentiation may be facilitated. Specifically, the nanosatellite structure may be provided to be spaced apart from one side of the substrate, the first linker may be compressed by applying a magnetic field to the other side of the substrate, and the nanosatellite structure may move in a direction toward the substrate. In this case, the nanosatellite structure may be fastened closely to the substrate, and stem cell adhesion and differentiation may be facilitated.

In addition, specifically, the nanosatellite structure may be provided to be spaced apart from one side of the substrate, the first linker may be stretched by applying a magnetic field to the upper side of the nanosatellite structure, which is one side of the substrate, and the nanosatellite structure may move in a direction away from the substrate. In this case, the nanosatellite structure may move away from the substrate, and stem cell adhesion and differentiation may be facilitated.

The magnetic field may be applied from outside the body to remotely control the nanosatellite-substrate complex in the body.

The magnetic field may be applied at a strength of 100 mT to 500 mT.

According to another aspect of the present invention, one embodiment of the present invention may include a method for preparing a nanosatellite-substrate complex for regulating stem cell adhesion and differentiation, the method comprising: coating the surface of iron oxide with a silica having an amine group to form magnetic particles; providing gold nanoparticles on the surfaces of the magnetic particles; adding and dispersing the magnetic particles having the gold nanoparticles provided thereon in a solution containing a polymer linker to form a first linker and a second linker; reacting the first linker with a substrate having amine groups formed thereon, so that the first linker is bound to at least a portion of the amine groups formed on the substrate and the magnetic particles are conjugated to the substrate; deactivating amine groups, which remain unbound to the first linker on the substrate, by treatment with a deactivating group; and conjugating a ligand to the second linker.

For preparation of the magnetic particles, iron chloride hydrate, sodium acetate and DI water may be mixed and stirred with ethylene glycol and washed to form a core composed of iron oxide, and the core may be coated with a shell comprising silica by adding 3-aminopropyl triethoxysilane (APTES) thereto. The surface of the magnetic particle may comprise at least one of an amino group (—NH₂) and a thiol group (—SH).

The surface of the magnetic particle may have gold nanoparticles conjugated thereon.

The gold nanoparticles may have any one of a first average diameter, a second average diameter, and a third average diameter. The first average diameter of the gold nanoparticles may be 3.5 nm to 10.5 nm, the second average diameter may be 12 nm to 14 nm, and the third average diameter may be 15 nm to 25 nm.

When the gold nanoparticles have the first average diameter, first gold seed particles may be provided on the surfaces of the magnetic particles and then grown into first gold nanoparticles on the surfaces of the magnetic particles. The first gold seed particles may be provided on the surface of the magnetic particle by reaction with a functional group on the surfaces of the magnetic particles, and the magnetic particles having the first gold seed particles provided thereon may be added to and stirred in a gold-containing solution to grow the first gold seed particles into gold nanoparticles. The first gold seed particles may be 3.0 nm to 5.0 nm in average diameter.

In addition, when the gold nanoparticles have any one of the second average diameter and the third average diameter, second gold seed particles may be grown and then conjugated to the magnetic particles. The second gold seed particles may be added to a gold-containing solution and grown into second gold nanoparticles by heating and stirring. Then, the second gold nanoparticles may be provided on the surfaces of the magnetic particles by reaction with an amine group on the surfaces of the magnetic particles. The second gold seed particles may have an average diameter of 3.0 nm to 13 nm.

The gold-containing solution may comprise a first solution containing sodium citrate (Na₃Cit) and a second solution containing chloroauric acid (HAuCl₄). The first solution and the second solution may be sequentially added, and the size of the gold nanoparticles may be controlled by controlling the number of times the first solution and the second solution are added. Specifically, as the number of times the first solution and the second solution are added increases, the size of the gold nanoparticles formed may increase.

The concentration of the first solution may be 40 mM to 80 mM, and the concentration of the second solution may be 5 mM to 30 mM.

The deactivating group for deactivating the substrate may have a structure of Formula 2. n in Formula 2 may be a number ranging from 113 to 450, preferably from 113 to 225.

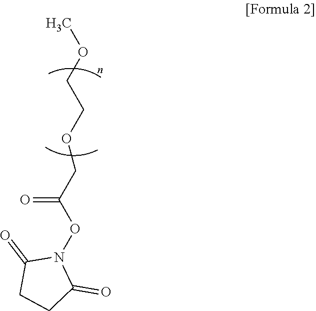

In addition, the present invention may include a method of regulating stem cell adhesion and differentiation using the nanosatellite-substrate complex, the method comprising regulating stem cell adhesion and differentiation by applying a magnetic field to the nanosatellite-substrate complex having the above-described characteristics.

The magnetic field may be applied from outside the body to remotely control the nanosatellite-substrate complex in the body.

The magnetic field may be applied at a strength of 100 mT to 500 mT.

The nanosatellite structure may be provided to be spaced apart from one side of the substrate, and a plurality of the nanoassemblies may be provided adjacent to each other on the surface of the magnetic particle. When the gold nanoparticles have an average diameter of 3.5 nm to 10.5 nm (first average diameter) and the distance between the gold nanoparticles in the nanoassemblies provided adjacent to each other is 3 nm to 4 nm (first distance), the first linker may be stretched by applying the magnetic field to the upper side of the nanosatellite structure, which is one side of the substrate, and the nanosatellite structure may move in a direction away from the substrate to inhibit stem cell adhesion and differentiation. When the average diameter of the gold nanoparticles is the first average diameter and the distance between the adjacent gold nanoparticles is the first distance, stem cell adhesion and differentiation may be facilitated before application of the magnetic field, but stem cell adhesion and differentiation may be inhibited after application of the magnetic field.

The nanosatellite structure may be provided to be spaced apart from one side of the substrate, and a plurality of the nanoassemblies may be provided adjacent to each other on the surface of the magnetic particle. When the average diameter of the gold nanoparticles is 15 nm to 25 nm (third average diameter) and the distance between the gold nanoparticles in the nanoassemblies provided adjacent to each other is 18 nm to 22 nm (third distance), the first linker may be compressed by applying the magnetic field to the other side of the substrate, and the nanosatellite structure may move in a direction toward the substrate to facilitate stem cell adhesion and differentiation. When the average diameter of the gold nanoparticles is the third average diameter and the distance between the adjacent gold nanoparticles is the third distance, stem cell adhesion and differentiation may be inhibited before application of the magnetic field, but stem cell adhesion and differentiation may be facilitated after application of the magnetic field.

Hereinafter, examples and experimental examples of the present invention will be described. However, the following examples are only preferred examples of the present invention, and the scope of the present invention is not limited by the following examples.

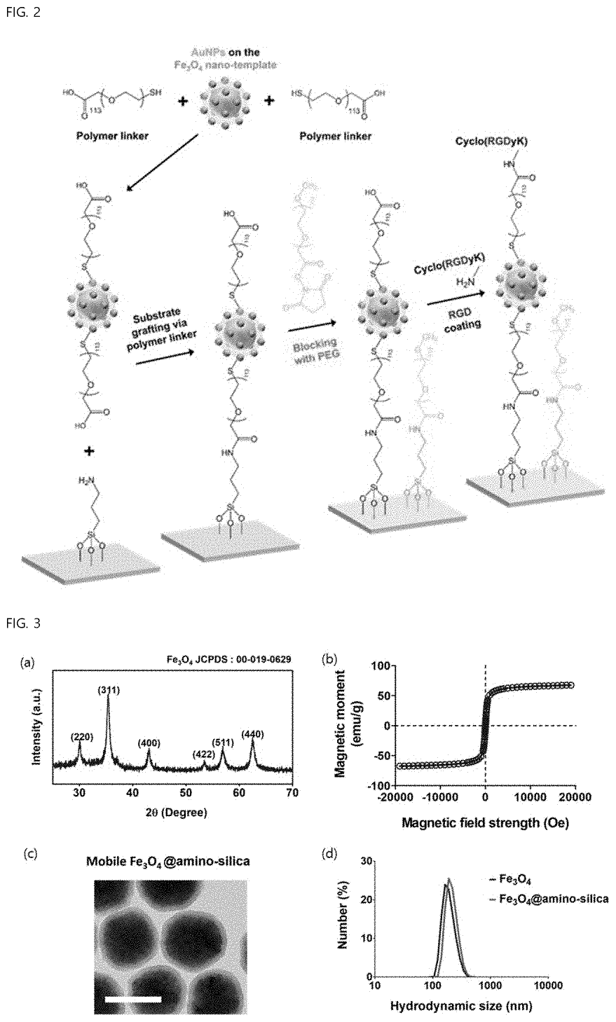

FIG. 2 schematically shows a process of preparing a nanosatellite-substrate complex in the following examples.

### Example 1

1. Preparation of Magnetic Particles

(1) Core Preparation

0.541 g (2 mmol) of iron (III) chloride hexahydrate (FeCl₃6H₂O), 0.492 g (6 mmol) of sodium acetate, and 2.703 mL (150 mmol) of deionized (DI) water were mixed with 50 mL of ethylene glycol. Subsequently, the mixture was mechanically stirred for 3.5 hours at 200° C. to obtain iron oxide (Fe₃O₄). The iron oxide was then washed successively three times with ethanol and resuspended in ethanol.

(2) Preparation of Magnetic Particles by Shell Formation

3 mL of ammonia solution and 9 mL of DI water were added to and mixed with 30 mg of the iron oxide core (prepared in (1) above) dispersed in 60 ml of ethanol. The mixture was shaken vigorously for 15 minutes, and then added to 60 μL of tetraethyl orthosilicate (TEOS). Subsequently, the suspension was stirred for 1.5 hours, washed successively three times with ethanol, and then suspended in 12 mL of ethanol. 10 mL of 2-propanol was added to 4 mL of this suspension and sonicated for 10 minutes. 0.1 mL of (3-aminopropyl)triethoxysilane was added to this suspension, followed by mechanical stirring at 80° C. for 3 hours, thus forming magnetic particles having a shell comprising silica on the iron oxide core and an amino group formed thereon. The magnetic particles were then washed successively three times with ethanol followed by washing successively three times with DI water and resuspended in 4 mL of DI water.

2. Formation of Gold Nanoparticles on Magnetic Particle Surface

(1) Preparation of Gold Seed Particles

A solution containing gold seed particles having a diameter of 3.5 nm were prepared by mixing 2.5 mL of DI water containing 5 mM gold(III) chloride trihydrate (HAuCl₄·3H₂O) and 2.5 mL of DI water containing containing 5 mM trisodium citrate dihydrate (Na₃Cit), followed by adding the mixture to 45 mL of DI water and then vigorously stirring for 10 min.

0.1 M sodium borohydride in 1.5 mL of DI water was rapidly injected into the solution containing gold seed particles having a diameter of 3.5 nm to trigger a reduction reaction, followed by stirring for another 10 min. When the gold nanoparticle solution changed from yellow to orange-red color, it was stabilized for 6 hours and stored at 4° C.

(2) Formation of Gold Nanoparticles on Magnetic Particle Surface

1) To form gold nanoparticles having a diameter of 6.5 nm on the magnetic particle surface, the 3.5-nm gold seed particles prepared in 2-(1) above were conjugated to the surfaces of the magnetic particles and then further grown.

First, 4 mL of a solution containing the magnetic particles prepared in 1-1 above was mixed with 25 mL of a solution containing the 3.5-nm-diameter gold seed particles prepared in 1-2-(1) above, and the mixture was stirred at 25° C. overnight, and washed three times in succession with DI water using a permanent magnet, thereby coating the surfaces of the magnetic particles with the 3.5-nm-diameter gold seed particles. The magnetic particles coated with the gold seed particles were stabilized in 10 mL of DI water containing 1 wt % of polyvinylpyrrolidone (PVP; weight-average molecular weight: 55,000 Da) for 3 hours, and then dispersed in 20 mL of DI water.

The dispersion and 200 mL of DI water containing 1 wt % of PVP were mixed with 4 mL of DI water containing L-ascorbic acid, followed by vigorous stirring for 5 minutes. While the solution was stirred, 1 mL of DI water containing 5 mM HAuCl₄·3H₂O was added, followed by stirring for an additional 10 min. The process of adding 1 mL of DI water containing 5 mM HAuCl₄·3H₂O and stirring for 10 minutes was performed once, whereby the average diameter of the gold nanoparticles conjugated to the magnetic particle surface was grown to 6.5 nm, thereby preparing a material in which the distance between adjacent gold nanoparticles on the magnetic particle surface was 3.5 nm. Thereafter, the material was washed three times in succession with DI water using permanent magnetism, and dispersed in 10 mL of DI water.

2) A material was formed in which the distance between adjacent gold nanoparticles having a diameter of 6.5 nm on the magnetic particle surface was 18.5 nm.

4 mL of a solution containing the magnetic particles prepared in 1-1 above was mixed with 5 mL of a solution containing the 3.5-nm-diameter gold seed particles prepared in 1-2-(1) above, and the mixture was stirred at 25° C. overnight, and washed three times in succession with DI water using a permanent magnet, thereby coating the surfaces of the magnetic particles with the 3.5-nm-diameter gold seed particles. The magnetic particles coated with the gold seed particles were stabilized in 10 mL of DI water containing 1 wt % of polyvinylpyrrolidone (PVP, weight-average molecular weight: 55,000 Da) for 3 hours, and then dispersed in 20 mL of DI water.

0.5 mL of DI water containing 5 mM HAuCl₄·3H₂O, 0.5 mL of DI water containing 200 mM potassium iodide (KI), 40 mL of DI water containing 1 wt % PVP, and 3 mL of DI water containing 20 mM L-ascorbic acid were mixed together and stirred at 25° C. for 30 min. The solution was mixed with 20 mL of a solution containing the 3.5-nm-diameter gold seed particle-conjugated magnetic particles, followed by shaking for 1 hour. The solution was then washed successively three times with DI water using a permanent magnet and suspended in 10 mL of DI water.

3. Conjugation of Linker to Gold Nanoparticles

The gold nanoparticle-conjugated magnetic particles were PEGylated with an elastic and bendy polymer linker.

DI water containing 1 mL of the gold nanoparticle-conjugated magnetic particles was mixed with 1 mL of 0.01% sodium dodecyl sulfate solution. The resulting mixture solution was further mixed with 1 mM of carboxymethyl-PEG-thiol, weight-average molecular weight: 5,000 Da; Laysan Bio, Inc.; catalog number: CM-PEG-SH-5000) as a polymer linker and then stirred for 3 hours to coat the gold nanoparticles with the PEG linker via gold-thiol bonding. Then, the mixed solution was washed successively three times with DI water and suspended in 1 mL of 50 mM 2-ethanesulfonic acid buffer.

4. Conjugation of First Linker to Substrate and Substrate Deactivation

To conjugate the material prepared in 1-3 above to substrates, culture grade glass coverslips (8×8 mm) were prepared as the substrates. First, for amine-activation of the substrate, the substrates were soaked in a mixed solution of 3-aminopropyltriethoxysilane (APTES) and ethanol (1:19) for 12 hours in the dark. Next, the amine-activated substrates were washed successively three times with ethanol and successively three times with DI water. For the EDC/NHS activation of the PEG linker, the material prepared in 1-3 above was stirred for 1 hour after mixing with 0.05 mL of DI water containing 20 mM N-(3-dimethylaminopropyl)-N′-ethylcarbodiimide hydrochloride (EDC) and 0.05 mL of DI water containing 20 mM N-hydroxysulfosuccinimide (NHS). Subsequently, the EDC/NHS-activated material was suspended in 10 mL of DI water to conjugate it to the amine-activated substrates by the PEG linker. Next, the reaction was carried out for 70 minutes in order to keep the density of the conjugated ligand constant.

Thereafter, DI water and phosphate-buffered saline were serially used to rinse the substrate.

Then, in order to allow stem cells to specifically bind to the ligand, the unreacted amine group on the substrate was deactivated. To this end, 2 mL of DI water containing methoxy-PEG-succinimidyl-carboxymethyl-ester (weight-average molecular weight: 5,000 Da; JenKem; catalog number: M-SCM-5000) was reacted with the substrate in the dark for 2 hours, followed by washing successively three times with DI water.

5. Conjugation of Ligand to Second Linker

The resulting material was reacted with 0.1 mL of PBS containing 1 mM RGD peptide [Cyclo(-RGDyK); AS-61183-5 from AnaSpec, Inc.] including an amine-bearing lysine residue, followed by shaking overnight in the dark, thereby providing the RGD ligand on the gold nanoparticle surface.

Example 1 was prepared as follows by tuning the diameter of gold nanoparticles and the distance between adjacent gold nanoparticles.

TABLE 1

| | | Distance between | |
|---|---|---|---|
| | Average diameter of | gold | Expressions |
| | gold nanoparticles | nanoparticles | in figures |
| Example 1-1 | 6.5 nm | 3.5 nm | S(6.5, 3.5) |
| Example 1-2 | 6.5 nm | 18.5 nm | S(6.5, 18.5) |

### Example 2

Example 2 was prepared in the same manner as in Example 1, except that the average diameter of gold nanoparticles was 13 nm. In Example 2, gold nanoparticles were prepared in the following way. In the nanosatellite-substrate complex prepared in Example 2, the average diameter of gold nanoparticles was 13 nm and the distance between adjacent gold nanoparticles was about 17 nm.

The gold nanoparticles having a diameter of 13 nm were prepared by adding 100 Ml of DI water containing 1 mM HAuCl₄·3H₂O to the solution, followed by stirring at 100° C. for 20 minutes. Finally, 10 mL of DI water containing 38.8 mM Na₃Cit was rapidly added to the mixture solution, followed by stirring for 10 min. When the gold nanoparticle solution changed from yellow to burgundy red in color, it was suspended and stabilized in 100 mL of DI water containing 1 mM Na₃Cit.

8 mL of a solution containing 13-nm-diameter gold nanoparticles was mixed with 4 mL of a solution containing the magnetic particles, followed by stirring at 25° C. overnight. After the 13-nm-diameter gold nanoparticles were conjugated to the magnetic particles, they were washed successively three times with DI water using a permanent magnet. The washed product was stabilized in 10 mL of DI water containing 1% PVP solution for 3 hours and then dispersed in 10 mL of DI water.

After EDC/NHS activation of the PEG linker conjugated to the gold nanoparticles, the reaction was carried out for 55 minutes in order to make the density of the conjugated ligand constant.

### Example 3

Example 3 was prepared in the same manner as in Example 1, except that the average diameter of gold nanoparticles was 20 nm. In Example 3, gold nanoparticles were prepared in the following manner. In the nanosatellite-substrate complex prepared in Example 3, the average diameter of gold nanoparticles was 20 nm and the distance between adjacent gold nanoparticles was about 20 nm.

Gold nanoparticles with a size of 20 nm were prepared using gold nanoparticles with a size of 13 nm as gold seed particles. 4 mL of a solution containing 13-nm gold seed particles was diluted in 88 mL of DI water, followed by stirring at 90° C. for 20 minutes. 0.4 mL of DI water containing 25 mL of HAuCl₄·3H₂O and 0.8 mL of DI water containing 60 mM Na₃Cit were added to the diluted gold seed particle solution, followed by stirring for 30 minutes. Thereafter, the solution changed to a burgundy-purple color, signifying that the gold seed particles were grown to form gold nanoparticles. Finally, the formed gold nanoparticles with a size of 20 nm were suspended in 100 mL of DI water containing 1 mM Na₃Cit.

8 mL of a solution containing 20-nm-diameter gold nanoparticles was mixed with 4 mL of a solution containing the magnetic particles, followed by stirring at 25° C. overnight. After the 20-nm-diameter gold nanoparticles were conjugated to the magnetic particles, they were washed successively three times with DI water using a permanent magnet. The washed product was stabilized in 10 mL of DI water containing 1% PVP solution, and then dispersed in 10 mL of DI water.

After EDC/NHS activation of the PEG linker conjugated to the gold nanoparticles, the reaction was carried out for 40 minutes in order to make the density of the conjugated ligand constant.

### Example 4

Example 4 was prepared in the same manner as in Example 1, except that the gold nanoparticles were prepared so as to cover the entire outer surface of the magnetic particles. In Example 4, gold nanoparticles were prepared in the following way.

3 mL of DI water containing 5 mM HAuCl₄·3H₂O and 197 mL of DI water containing 1.8 M potassium carbonate were mixed together and stirred at 25° C. for 12 hours. This solution was mixed with 0.125 mL of DI water containing 5 wt % PVP and 0.125 mL of DI water containing 3.7 wt % formaldehyde, and then added to 2 mL of 3.5-nm-diameter gold nanoparticle-conjugated magnetic particles successively ten times at 2-min intervals, followed by shaking for 1 hour.

The solution was rinsed successively three times with DI water using a permanent magnet and dispersed in 1 mL of DI water. The solution changed from translucent reddish-orange to bluish-purple in color, and thus the product was prepared.

### Example 5

Nanosatellite-substrate complexes of Example 5 were prepared in the same manner as in Example 2, except that gold nanoparticles were grown to an average diameter of 12.3±0.6 nm and the density of the nanoassemblies conjugated to the magnetic particle surface was varied.

TABLE 2

| | | Expression |
|---|---|---|
| | Nanoassembly density | in figures |
| Example 5-1 | 39 ± 5/magnetic particle | low |
| Example 5-2 | 109 ± 6/magnetic particle | moderate |
| Example 5-3 | 180 ± 13/magnetic particle | high |

### COMPARATIVE EXAMPLES

Comparative Example 1: Only the substrate was prepared without the magnetic particles, the nanoassemblies and the first linker.

Comparative Example 2: Nanosatellite-substrate complexes were prepared in the same manner as in Example 5, except that the RGD ligand was not used.

TABLE 3

| | Nanoassembly density |
|---|---|
| Comparative Example 2-1 | 39 ± 5/magnetic particle |
| Comparative Example 2-2 | 109 ± 6/magnetic particle |
| Comparative Example 2-3 | 180 ± 13/magnetic particle |

Comparative Example 3: Nanosatellite-substrate complexes were prepared by changing the RGD ligand to the RAD ligand.

TABLE 4

| | Nanoassembly density |
|---|---|
| Comparative Example 3-1 | 39 ± 5/magnetic particle |
| Comparative Example 3-2 | 109 ± 6/magnetic particles |
| Comparative Example 3-3 | 180 ± 13/magnetic particles |

Comparative Example 4: Nanosatellite-substrate complexes without the RGD ligand were prepared by tuning the diameter of gold nanoparticles and the distance between adjacent gold nanoparticles.

TABLE 5

| | | Distance between | |
|---|---|---|---|
| | Diameter of gold | adjacent gold | |
| | nanoparticles | nanoparticles | |
| Comparative Example 4-1 | 6.5 nm | 3.5 | nm |
| Comparative Example 4-2 | 13 nm | 16.5 | nm |
| Comparative Example 4-3 | 20 nm | 20 | nm |

Comparative Example 5: Nanosatellite-substrate complexes were prepared by tuning the diameter of gold nanoparticles and the distance between adjacent gold nanoparticles and using the RAD ligand instead of the RGD ligand.

TABLE 6

| | | Distance between | |
|---|---|---|---|
| | Diameter of gold | adjacent gold | |
| | nanoparticles | nanoparticles | |
| Comparative Example 6-1 | 6.5 nm | 3.5 | nm |
| Comparative Example 6-2 | 13 nm | 16.5 | nm |
| Comparative Example 6-3 | 20 nm | 20 | nm |

The nanosatellite-substrate complexes prepared in the Examples and the Comparative Examples were analyzed, and an experiment was conducted on whether stem cell adhesion and differentiation could be regulated by applying a magnetic field to the nanosatellite-substrate complex.

In Experimental Examples and the drawings for the Experimental Examples, the nanosatellite-substrate complex was expressed as S (small), M (medium), or L (large) depending on the size of the gold nanoparticles. “S” is a case in which the 6.5-nm gold nanoparticles prepared in Example 1 were applied, “M” is a case in which the 13-nm gold nanoparticles prepared in Example 2 were applied, and “L” is a case in which the 20-nm gold nanoparticles prepared in Example 3 were applied.

Also, in the Experimental Examples and the drawings for the Experimental Examples, the nanosatellite-substrate complex was expressed as (X, Y). Here, X is the size of the gold nanoparticles, and Y is the distance (nm) between adjacent gold nanoparticles. When X is 200 nm and Y is 0 nm, it means that the nanoassembly is provided so as to completely cover the outer surface of the magnetic particle.

In addition, in the case in which the diameter of gold nanoparticles was maintained constant and only the density of the nanoassemblies conjugated to the magnetic particle was tuned, a nanoassembly density of 39±5/magnetic particle was expressed as “low ligand-nanoassembly density”, “low”, or “low ligand density”. A nanoassembly density of 109±6/magnetic particle was expressed as “moderate ligand-nanoassembly density”, “moderate”, or “moderate ligand density”. A nanoassembly density of 180±13/magnetic particle was expressed as “high ligand-nanoassembly density”, “high”, or “high ligand density”.

In addition, a case in which no magnetic field was applied was expressed as OFF, or whether or not a magnetic field was applied was not expressed. A case, in which the first linker was compressed by applying a magnetic field to the other side of the substrate and the nanosatellite structure was moved in a direction toward the substrate, was expressed as “fastened”, “fas”, “Falling” ON, “the nanosatellite structure is conjugated”, or “the nanosatellite structure is fastened”. A case, in which the first linker was stretched by applying a magnetic field to the upper side of the nanosatellite structure corresponding to one side of the substrate and the nanosatellite structure was moved in a direction away from the substrate, was expressed as “unfastened”, “unfas”, “Rising” ON, “the nanosatellite structure becomes loose”, or “the nanosatellite structure is unfastened”.

### Experimental Methods

1. X-Ray Diffraction (XRD)

To verify crystalline phases including both iron oxide (Fe₃O₄) and gold nanoparticles in the Example, XRD (D/MAX-2500V/PC, Rigaku) analyzation using Cu Kα radiation with the diffraction spectrum in the range of 25°<20<70° was performed. Reference data for the Fe₃O₄ and Au phases were exploited to index peaks.

2. Vibrating Sample Magnetometry (VSM)

For the magnetic property analysis of magnetic particles and nanosatellite structures, VSM measurement (EV9-380, Microsense) was carried out at 25° C. with an applied magnetic field strength of −19,000 to 19,000 Oe. The resultant magnetic moments after normalization to the dry weight of the samples were displayed in reversible hysteresis loops.

3. Transmission Electron Microscopy (TEM)

TEM imaging (Tecnai 20, FEI, USA) was conducted to characterize the size, shape, and monodispersity of the gold seed particle-mediated-grown gold nanoparticles in suspension, gold seed particle-mediated-grown gold nanoparticles on magnetic particles, and magnetic particles coated with amine-activated silica shells. Tunable gold nanoparticles on magnetic particles were analyzed to quantify the diameter and edge-to-edge inter-distance of adjacent gold nanoparticles using five different images via ImageJ software.

4. Dynamic Light Scattering (DLS) Examination

The hydrodynamic diameter distributions of nanosatellite structures, gold nanoparticles, iron oxide cores before silica shell coating and magnetic particles after silica shell coating were determined via DLS measurements: “Small” (6.5, 3.5)-, “Medium” (13, 17)-, and “Large” (20, 20)-sized RGD groups with the following notations: (RGD-bearing gold nanoparticle diameter, gold nanoparticle edge-to-edge RGD distance).

5. UV-Vis Spectroscopy Analysis

UV-Vis spectroscopy (Shimadzu UV-1800) was performed in the wavelength range of 350 to 800 nm to obtain absorption spectra of the gold nanoparticles following gold seed particle-mediated growth and tunable gold nanoparticle arrangements on magnetic particle surfaces.

6. High Angle Annular Dark Field-Scanning Transmission Electron Microscopy (HAADF-STEM)

HAADF-STEM imaging (Cs-corrected JEM ARM200CF probe, JEOL Ltd.) at 200 kV under spherical aberration (C3) of 0.5 to 1 μm was carried out to identify successful tuning of the gold nanoparticle diameter and the distance between gold nanoparticles. The measured phase, convergence semi-angle, and collection semi-angle were 27 to 28, 21, and 90 to 370 mrad, respectively. A JEOL defined electron probes with sizes of 8 C and 9 C corresponding to 1.28 and 1.2 Å, respectively, a 2048×2048-pixel area with a pixel dwelling time of 10 to 15 μs, a probe current range of 10 to 20 pA, and an emission current of 8 to 13 μA were used for the imaging. The magnetic particles and uniformly distributed gold nanoparticles were depicted by gray and white colors in the HAADF-STEM images, respectively. Eight different HAADF-STEM images were used to quantify the gold nanoparticle diameter, gold nanoparticle edge-to-edge distance, the number of the gold nanoparticles per magnetic particle, and the total surface area of the gold nanoparticles per magnetic particle via Image J software.

7. Energy-Dispersive X-Ray Spectroscopy (EDS) Mapping

EDS mapping using two SDD detectors while applying identical conditions used for the HAADF-STEM imaging was carried out to confirm the dual presence of the Fe and Au elements in the magnetic particles and gold nanoparticles, respectively, in the nanosatellite structures.

8. High Resolution-Scanning Transmission Electron Microscopy (HR-STEM)

HR-STEM imaging at the atomic scale was performed to probe the atomic arrangements of the crystalline phases of the magnetic particles and gold nanoparticles used to construct the nanosatellite structures. The other settings were identical to the conditions used for the HAADF-STEM imaging, except for the probe (Cs-corrected JEM ARM200C) with 2.5-5.0 million magnifications at 200 kV. The average lattice spacing of the gold nanoparticle was computed and labeled from the periodic lattice fringes to verify its crystalline structure. The average unit cell lattice parameter for the magnetic particle was computed and labeled to confirm its crystalline structure.

9. Fourier Transform Infrared Spectroscopy (FTIR)

FTIR analysis was carried out using Nicolet iS10 from Thermo Fisher Scientific to characterize the consecutive changes in chemical bonds after the stabilization of the gold nanoparticle arrangements on the magnetic particles with PVP, the conjugation of PEG to the gold nanoparticle arrangements on the magnetic particles, and the nanosatellite structures. To prepare the sample, the suspension was first dried and then densely packed into KBr pellets. The corresponding absorption peaks along with serial changes in chemical bonds were indexed.

10. Field Emission-Scanning Electron Microscopy (FE-SEM)

FE-SEM imaging (FEI, Quanta 250 FEG) was conducted to verify the even distribution of the magnetic particles exhibiting a tunable nanoassembly conjugated to the substrate via the PEG linker. The samples were prepared by drying them at 37° C. Prior to FE-SEM imaging, the samples were sputter-coated with platinum for 90 seconds. The number of substrate-conjugated magnetic particles per μm² was counted using six different FE-SEM images, the results of which were invariant across all of the groups. The surface-grafted nanoassembly density was obtained by multiplying the number of substrate-conjugated magnetic particles per μm² by total surface areas of nanoassemblies per magnetic particle.

11. Integrin β1 Binding and Clustering to Nanoassemblies

The nanosatellite-substrate complexes were placed in PBS including 50 μg/mL of integrin β1 at 4° C. for 12 hours to evaluate the binding efficiency of integrin β1 to the nanoassemblies. After the binding process, 4% (w/v) paraformaldehyde (PFA) at 25° C. was used to fix the bound and clustered integrin β1 on the substrate for 10 minutes and then subjected to immunofluorescence staining for integrin β1. Confocal microscopy imaging was utilized to examine the integrin β1 binding efficiency by quantifying the immunofluorescence intensity in six different images using a histogram function.

12. Integrin Clustering Analysis of Stem Cells Via Gold-Immunolabeling

Gold-immunolabeling was performed to probe integrin binding to and clustering on the RGD ligand using 40 nm gold nanoparticles (hereinafter referred to 40-nm gold nanoparticles) coated with secondary antibodies. The gold nanoparticles used here are larger in diameter than those of the gold nanoparticles in the “S” (6.5 nm), “M” (13 nm), and “L” (20 nm) nanoassemblies.

To synthesize the 40-nm gold nanoparticles, 50 mL of DI water containing 1 mM HAuCl₄·3H₂O was first boiled and then vigorously stirred for 15 minutes. Next, the solution was mixed with 5 mL of DI water containing 1% (w/v) sodium citrate and boiled for another 15 minutes. After boiling, the mixture was cooled down to 25° C., which yielded 40 nm spherical gold nanoparticles. The resulting 40-nm gold nanoparticles were incubated in secondary antibody [goat anti-mouse (H+L) IgG (1:100), Abcam] with gentle shaking at 37° C. for 16 hours. After incubation, DI water was used to wash the sample.

For the gold-immunolabeling, stem cells were initially cultured on nanosatellite-substrate complexes in growth medium. After culturing, 0.1 M 1,4 piperazine bis(2-ethanosulfonic acid) (PIPES) buffer (pH=7.4) was used to wash the stem cell-adhered nanosatellite-substrate complexes for 2 min, which were then fixed with 4% PFA for 15 min followed by washing successively three times with PBS. Next, a buffer (pH=7.2) composed of DI water including sucrose, NaCl, MgCl₂, HEPES, and 0.5% Triton X-100 was applied to the fixed stem cells for 1 min to permeabilize them. Subsequently, the substrates were blocked with the blocking buffer (0.1 M PIPES buffer including 1% BSA and 0.1% Tween 20, pH=7.4) for 1 hour to minimize non-specific binding to the 40-nm gold nanoparticles.

Next, the substrates were immersed in primary integrin β1 antibody (mouse) in blocking buffer and incubated at 37° C. for 2 hours, followed by washing successively six times with 1% BSA solution for 2 min each. The substrates were then blocked for 15 min using 5% goat serum solution and then labeled with the 40-nm gold nanoparticles in suspension in PIPES buffer at 25° C. overnight. Once the integrin β1 labeling with the 40-nm gold nanoparticles was completed, PIPES buffer was used to rinse the substrates successively three times for 2 min.

After rinsing, 2.5% glutaraldehyde solution was used to permanently fix the cells for 5 min, followed by washing with PIPES for 3 min. To optimize the cell contrast in the FE-SEM images, the cells were suspended in PIPES buffer including 1% osmium tetroxide for 1 hour and were subsequently serially washed with PIPES buffer six times and then with DI water. Afterward, the cells were dried before FE-SEM imaging. Blue and yellow pseudo-colors were applied in the nanoscale gold-immunolabeling FE-SEM images to label the stem cells and 40-nm gold nanoparticles (indicating the clustered integrin β1), respectively. The number of 40-nm gold nanoparticles labeling the clustered integrin β1 per magnetic particles (colored in gray) was counted in six different FE-SEM images.

13. Confocal Immunofluorescence Imaging and Cell Analysis

The regulation of focal adhesion, mechanosensing, and differentiation of stem cells by tuning the RGD arrangements and RGD fastening was analyzed via confocal microscopy imaging after immunofluorescence staining. Initially, stem cell cultures were fixed with 4% PFA at 25° C. for 10 min and washed four times using PBS. Next, the fixed stem cells were blocked in blocking buffer (3% bovine serum albumin and 0.1% Triton-X in PBS) at 37° C. for 1 hour. The blocked cells were submerged in primary antibodies dissolved in blocking buffer at 4° C. and then washed four times with 0.5% (v/v) Tween 20 in PBS. Subsequently, the cells were incubated in blocking buffer including fluorescent secondary antibodies along with phalloidin at 25° C. for 40 min in the dark. After incubation, the samples were washed four times using 0.5% (v/v) Tween 20 in PBS and finally treated with DAPI antifade.

A confocal microscope (LSM700, Carl Zeiss) with equal exposure conditions was used to image the immunofluorescence-stained stem cells for all of the compared groups, which were then analyzed quantitatively through ImageJ software. The number of adhered stem cells was quantified by calculating the number of DAPI-stained cell nuclei per unit area, while the adhered cell area and aspect ratio (the length along the major/minor axis) were quantified using six different phalloidin-stained cell images. The number of focal adhesions was quantified through the counting of paxillin-positive clusters larger than 1 pmt. For the quantification of mechanotransduction (YAP) and the following differentiation (RUNX2) of stem cells, the nucleus/cytoplasm fluorescence ratio of stem cells was calculated to analyze their nuclear localization.

14. Western Blotting Imaging and Quantification of Stem Cell Differentiation

Western blotting was conducted to evaluate the efficacy of tuning the nanosatellite structures and nanosatellite fastening on stem cell differentiation.

First, stem cells were cultured in osteogenic induction medium on the nanosatellite-substrate complexes (“S”, “M”, and “L”) or remotely stimulated “L (Fastened)” group. After culturing, 400 μL of PRO-PREP™ protein extraction solution (iNtRON biotechnology) was applied to collect the total proteins from the stem cells, which were then preserved using 10 μL of protease inhibitor cocktail for 20 min after centrifugation at 4° C.

A BCA Protein Assay Kit (Thermo Scientific) was used to quantify the total protein concentration. The collected proteins mixed with loading dye were denatured via boiling at 100° C. for 8 min. The denatured proteins were separated by using 10% sodium dodecyl sulfate (SDS)-polyacrylamide gel electrophoresis (PAGE) (10%) at 110 V for 55 min, followed by transfer to polyvinylidene fluoride (PVDF) membranes at 120 V for 90 min. After 10 min of washing with 1× tris-buffered saline including 0.1% Tween 20 (TBST) buffer, the sample was blocked at 4° C. overnight using a blocking buffer (5% skimmed milk in TBST), followed by washing with TBST buffer. Next, the protein-adhered membranes were incubated in blocking buffer including primary antibodies for RUNX2 (60 kDa), ALP (75 kDa), and GAPDH (37 kDa) at 25° C. for 1 hour.

The membranes were washed successively three times for 5 min with TBST buffer and incubated in anti-HRP secondary antibody at 25° C. for 1 hour. ECL western blotting reagent (Immobilon Western Chemiluminescent HRP substrate, MERCK-Millipore) was used to label and process the membranes, while Linear Image Quant LAS 4000 mini chemiluminescent imaging system was employed to visualize the processed membranes. The protein expressions of RUNX2 and ALP were quantified after being normalized to GAPDH expression and presented.

15. In Situ Magnetic Atomic Force Microscopy Imaging (AFM)

To analyze magnetically induced fastening of the nanosatellite structures toward the substrate via tightening of a flexible polymer linker, in situ magnetic AFM imaging (Asylum Research, XE-100 Systems) was carried out. “L” group or remotely stimulated “L (Fastened)” group were used for this imaging in AC in air mode at room temperature using an SSS-SEIHR-20 AFM cantilever (Nanosensors) with a force constant of 5 to 37 N/m and a resonance frequency of 96 to 175 kHz. The imaging was conducted on a same area of the substrates for the “L (Fastened)” group with a permanent magnet (310 mT) placed in situ beneath the substrates to induce the fastening of the nanosatellite structures by attracting them downward toward the substrates and the “L” group with the magnet removed. Igor Pro 6.12A and ImageJ software were used to analyze the images exhibiting in situ contrast changes with corresponding quantification of height changes.

16. In Situ Magnetic Confocal Microscopy Imaging of Reversible Stem Cell Adhesion

To demonstrate the versatility of utilizing magnetic stimulation of the nanosatellite structures, magnetic fastening by placing a magnet for 4 hours underneath the substrates and subsequent unfastening by removing the magnet after 4 hours were tested to examine reversible stem cell adhesion. Real-time confocal microscopy imaging was conducted using a Nikon A1R confocal microscope mounted on a Nikon Eclipse Ti microscope equipped with a microscope stage incubator and Plan Apo VC 20× lens. Single cell was monitored via time-resolved snapshot imaging.

17. Statistical Analysis

All of the experiments conducted in the present invention were repeated independently at least twice. Graphpad Prism 8 software was exploited to perform all statistical quantitative evaluations. Two-tailed Student's t-tests were performed to analyze differences between two different groups without varying the time points while one-way analysis of variance (ANOVA) along with Tukey-Kramer post-hoc tests was exploited to compare multiple groups. Asterisks were assigned to p values with statistical significances tests (*: p<0.05; **: p<0.01; ***: p<0.001).

### Experimental Example 1

1. Characterization of Magnetic Particles

FIG. 3 shows the results of analyzing the characteristics of the magnetic particles prepared in the Example.

In FIG. 3(a), the presence of XRD peaks corresponding to the crystalline planes of Fe₃O₄ can be seen.

FIG. 3(b) shows VSM hysteresis curves obtained by measuring the reversible magnetic properties of Fe₃O₄. It can be seen that Fe₃O₄ has a high saturation magnetization value of 67.1 emu/g and nearly overlapping hysteresis curves.

FIG. 3(c) is a TEM image of the magnetic particles prepared by coating an Fe₃O₄ core with a silica shell having an amino group. The scale bar is 200 nm. It can be seen from the image that the core-shell structure was homogeneously formed.

FIG. 3(d) shows the results of DLS measurement of the hydrodynamic radius before and after coating the Fe₃O₄ core with the silica shell. It can be seen that the hydrodynamic radius was increased by the silica shell after coating. Here, the diameter of the magnetic particles after coating was 212±15 nm.

2. Characterization of Gold Nanoparticles

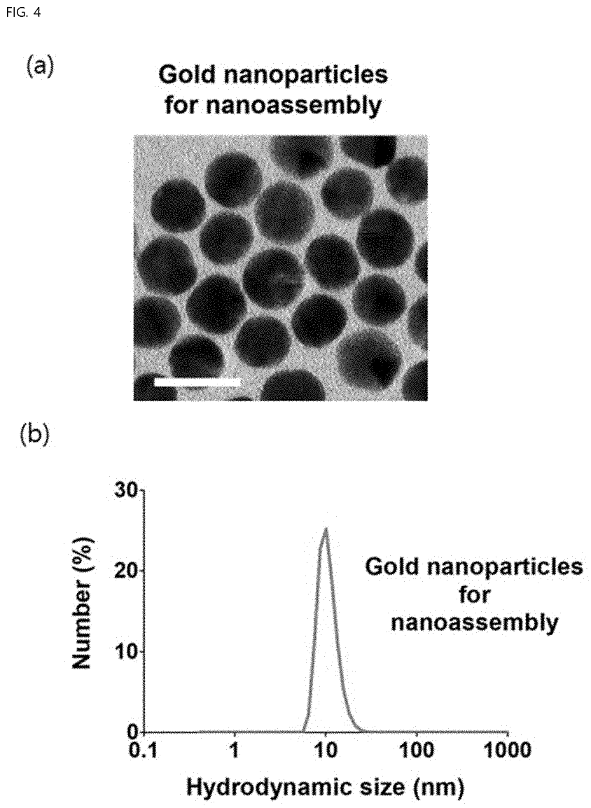

FIG. 4 shows the results of analyzing the characteristics of the gold nanoparticles prepared in the Example.

FIG. 4(a) is a TEM image showing the shape of gold nanoparticles. The scale bar is 20 nm. It can be seen that the shape and size of the gold nanoparticles were almost uniform.

FIG. 4(b) shows the results of examining the size distribution of gold nanoparticles using DLS. Here, the size of the gold nanoparticles was uniform (12.3±0.6 nm).

### Experimental Example 2

In order to investigate the effect of the distance between adjacent nanoassemblies conjugated to magnetic particles, nanosatellite-substrate complexes were prepared by varying the densities of nanoassemblies conjugated to magnetic particles. Although the density of nanoassemblies conjugated to each magnetic particle was varied, the overall amount of the nanoassemblies was maintained constant. The density of the nanoassemblies per magnetic particle was adjusted via an amine-gold reaction by optimizing the concentration ratio.

1. Characterization of Nanoassembly-Conjugated Magnetic Particles

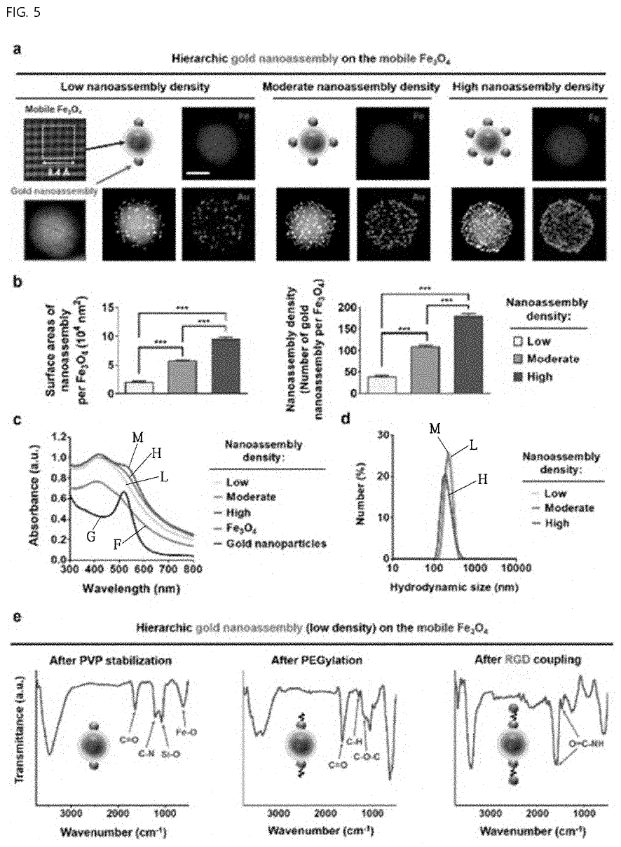

FIG. 5 shows the results of analyzing nanoassembly-conjugated magnetic particles.

FIG. 5(a) depicts HAADF-STEM images and energy dispersive X-ray spectroscopy (EDS), which show that the gold nanoparticles were conjugated to the magnetic particles at different densities. Here, the scale bar is 100 nm. The leftmost images show the atomic arrangements of crystalline phases of the magnetic particles and gold nanoparticle crystals. Here, the average lattice spacing between gold nanoparticles was measured to be 2.4 Å. In each image, purple represents iron (Fe), and green represents gold (Au). It can be confirmed that, as the density increased, the distribution density of gold (Au) increased.

FIG. 5(b) depicts graphs showing the results of quantifying the surface area and density of nanoassemblies conjugated to each magnetic particle (***: p<0.001). The surface area of the nanoassembly was measured to be 20494±3154, 57977±3345, and 95355±6733 nm2 at low, moderate and high nanoassembly group, respectively. The density of nanoassemblies was calculated as the number of nanoassemblies per magnetic particle, and was measured to be 39±5, 109±6, and 180±13 for low, moderate and high assembly groups, respectively.

FIG. 5(c) shows UV-Vis absorbance spectra of gold nanoparticles, magnetic particles (Fe₃O₄), and nanoassemblies at various densities. It can be confirmed that gold nanoparticles and magnetic particles were present in all the nanosatellite structures.

FIG. 5(d) shows the results of measuring DLS for nanoassemblies at various densities, and it is confirmed that the distribution of hydrodynamic sizes was similar between the nanoassemblies, even if the density of nanoassemblies conjugated to each magnetic particle was different.

FIG. 5(e) shows the results of FT-IR measurement for the nanosatellite structures (low) to which the nanoassemblies were conjugated at low density, after each of sequential PVP stabilization, linker tethering, and RGD ligand grafting.

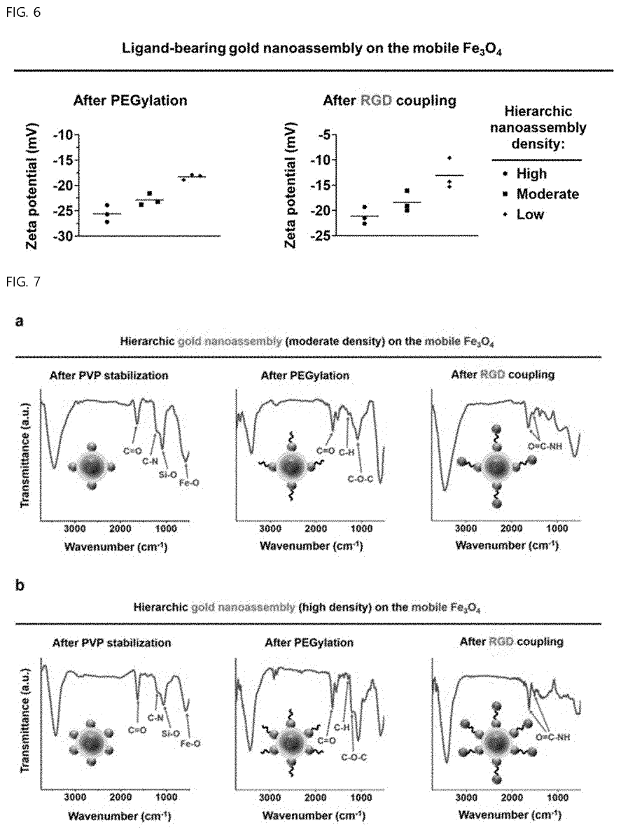

FIG. 6 shows the results of zeta potential analysis after conjugation of a PEG linker to gold nanoparticles conjugated to magnetic particles and further conjugation of an RGD ligand thereto (*: p<0.05, **: p<0.01, ***: p<0.001). It can be confirmed that, as the density of the nanoassembly decreased, the zeta potential approached zero, indicating that that the density of the nanoassembly was successfully tuned. Data are expressed as mean±standard error (n=3).

FIG. 7 shows the results of FT-IR analysis for each preparation step for nanosatellite structures (moderate) in which nanoassemblies were conjugated at moderate density and nanosatellite structures (high) in which nanoassemblies were conjugated at high density. Whether the nanosatellite structure was successfully synthesized was examined by identifying the functional groups in each step.

2. Examination of Movement of Nanosatellite-Substrate Complexes by Application of Magnetic Field

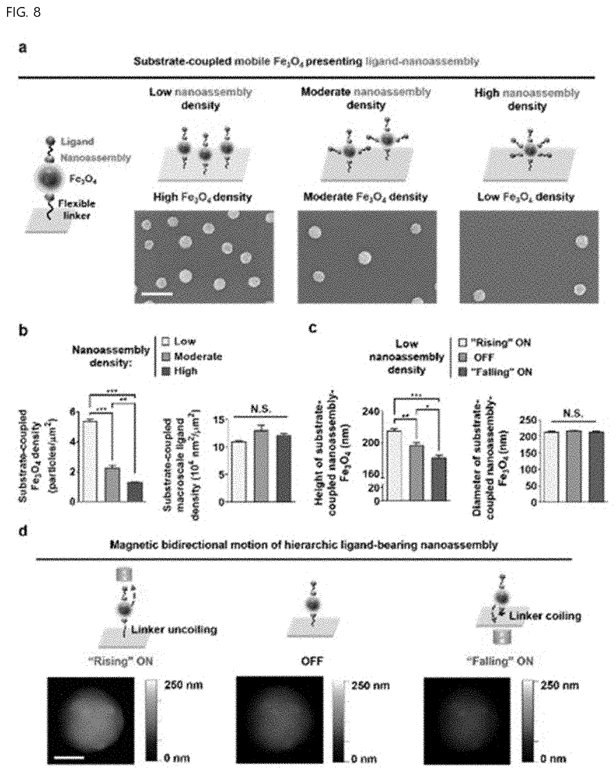

FIG. 8 shows the results of analysis of nanosatellite-substrate complexes formed by conjugating the nanosatellite structures to substrates via a linker.

FIGS. 8(a) and 8(b) show schematic views, SEM images, and quantification graphs after magnetic particles to which nanoassemblies were conjugated to substrates at different densities. The scale bar in the SEM images is 500 nm. Magnetic particles were conjugated to the substrates so that the overall density of the nanoassembly was constant. Therefore, it can be confirmed that, at low density, the number of the nanosatellite structures coupled to the substrate was larger, but moderate and high densities, the number of the nanosatellite structures coupled was smaller. The density of the nanosatellite structures coupled was calculated to be 5.4±0.2, 2.3±0.3 and 1.3±0.1 particles/μm² for low, moderate and high densities, respectively. However, the overall density of the nanoassembly on the substrate was calculated to be 110,600 to 131,300 nm²/μm², and was similar between all the three cases.

FIGS. 8(c) and 8 (d) show schematic views, AFM images, and quantified graphs, which show the results of analyzing the movement of the nanosatellite structures by applying a magnetic field to the nanosatellite-substrate complexes in different directions. A permanent magnet was placed at the upper side of the substrate, that is, the upper side of the nanosatellite structure so that the nanosatellite structure moved in a direction away from the substrate (“Rising” ON) via stretching of the linker, or a permanent magnet was placed under the lower side of the substrate so that the nanosatellite structure moved in a direction toward the substrate (“Falling” ON) via compression of the linker. In addition, measurements were made even in a state in which the nanosatellite structure did not move (OFF) because no permanent magnet was placed. Regardless of whether or not the magnetic field was applied, the diameter of the nanosatellite structure was measured to be 214.8 nm to 217.5 nm, and there was little difference in the diameter between Rising” ON, Falling” ON and OFF states. However, a difference in shading was observed in each AFM image, and it was confirmed that a difference in the height change of the nanosatellite structure occurred as the linker was compressed and stretched depending on the application of the magnetic field and the direction in which the magnetic field was applied. The height in each image was measured to be 215.3±3.5 nm for “Rising” ON, 180.7±4.7 nm for “Falling” ON and 196.3±6.1 nm for “OFF”. In FIG. 8(d), the scale bar is 100 nm.

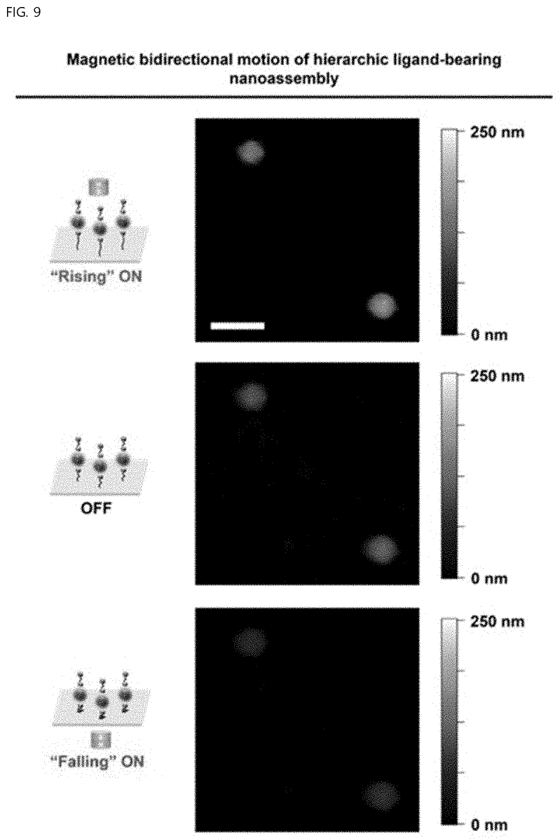

These results can also be found in the AFM images of FIG. 9. The scale bar in FIG. 9 is 400 nm. Therefore, it can be confirmed that the nanosatellite structure can be moved by compressing or stretching the linker through application of a magnetic field to the nanosatellite-substrate complex.

3. Experiment on Stem Cell Adhesion and Differentiation Using Nanosatellite-Substrate Complex

Examination was made as to whether the efficiency of binding of stem cells to the RGD ligand changed depending on the nanoassembly density. In the nanosatellite structure, the density of the nanoassemblies conjugated to each magnetic particle was varied, but the overall density of the nanoassemblies on the substrate was maintained constant. As stem cells, human mesenchymal stem cells (hMSCs) were used.

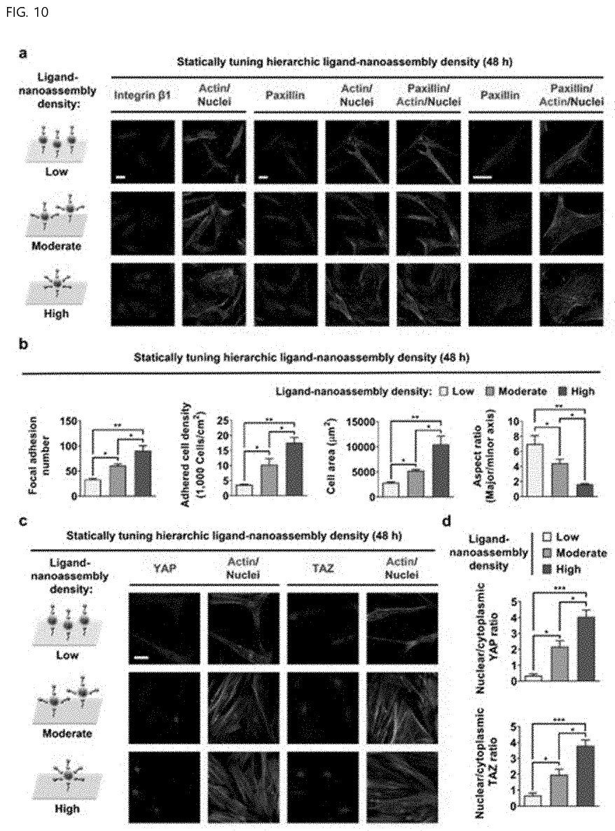

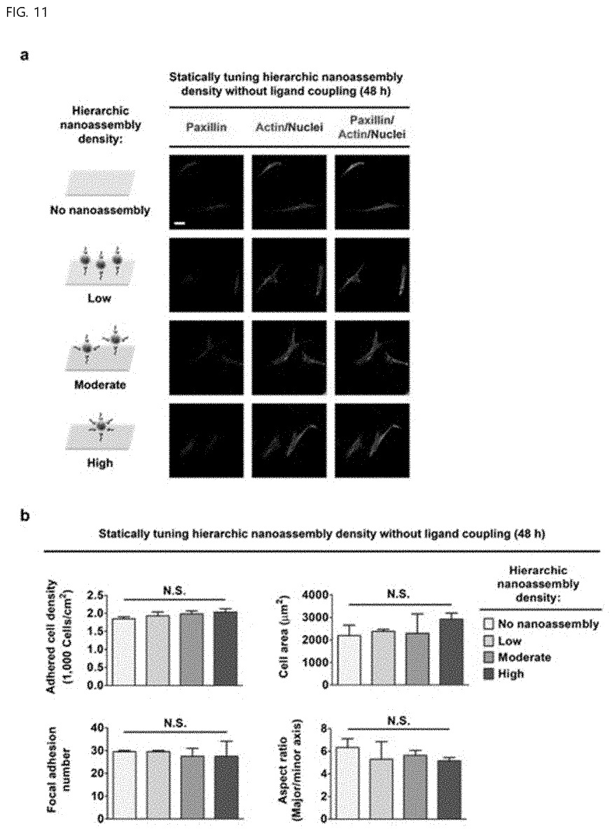

FIG. 10 depicts confocal immunofluorescence images and quantification graphs for stem cells cultured with nanosatellite-substrate complexes having different nanoassembly densities for 48 hours. Here, the scale bar is 50 μm, and the data are expressed as mean±standard error (n=20).

FIGS. 10(a) and 10(b) show confocal immunofluorescence images of actin/nucleus co-stained with integrin β1, paxillin, and F-actin and DAPI (nucleus) of adhered stem cells, and depict graphs showing the results of quantifying and analyzing the same. It can be confirmed that, as the nanoassembly density goes from low to high, more integrin β1 adhered to facilitate the development of paxillin and F-actin filaments to stimulate the spreading of stem cells.

FIGS. 10(c) and 10(d) show that, as the nanoassembly increased, focal adhesion of stem cells was stimulated to strengthen mechanosensing. This suggests that YAP and TAZ mechanotransducers further localize the nucleus.

Therefore, FIG. 10 proves that, as the nanoassembly density increases, stem cell adhesion and differentiation is facilitated.

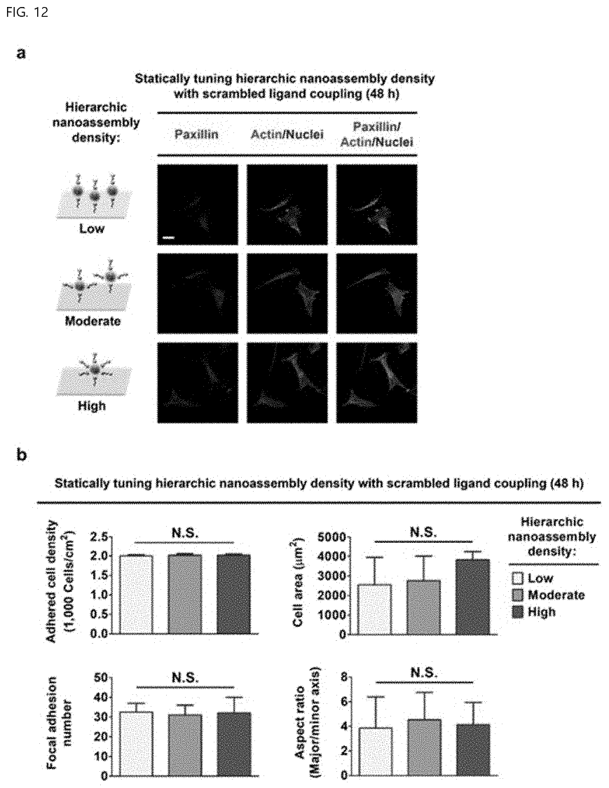

In addition, in order to evaluate the stem cell adhesion effect of the RGD ligand in the nanosatellite-substrate complex of the present invention, a nanosatellite-substrate complex without the RGD ligand (FIG. 11) and a nanosatellite-substrate complex comprising the RAD ligand instead of RGD ligand A complex (FIG. 12) was prepared and tested. In FIGS. 11 and 12, the scale bar is 50 μm, and data are expressed as mean±standard error (n=20).

Referring to FIG. 11, experiments were conducted on the case in which there was no nanoassembly and nanoparticle (that is, there was a substrate) and on nanosatellite-substrate complexes (low, moderate, and high) prepared by varying the density of nanoassemblies without the RGD ligand. It can be seen that, in all cases, the level of actin/nuclei co-stained with paxillin, F-actin, and DAPI was low.

In addition, it can be seen that FIG. 12 shows a nanosatellite-substrate complex comprising the RAD ligand, not the RGD ligand, and the same results as in FIG. 11 appear.

Therefore, it can be confirmed that the RGD ligand is essential for stem cell adhesion.

### Experimental Example 3

Nanosatellite-substrate complexes were prepared by varying the size of gold nanoparticles conjugated to magnetic particles while varying the distance between adjacent nanoassemblies (Examples 1 to 3).

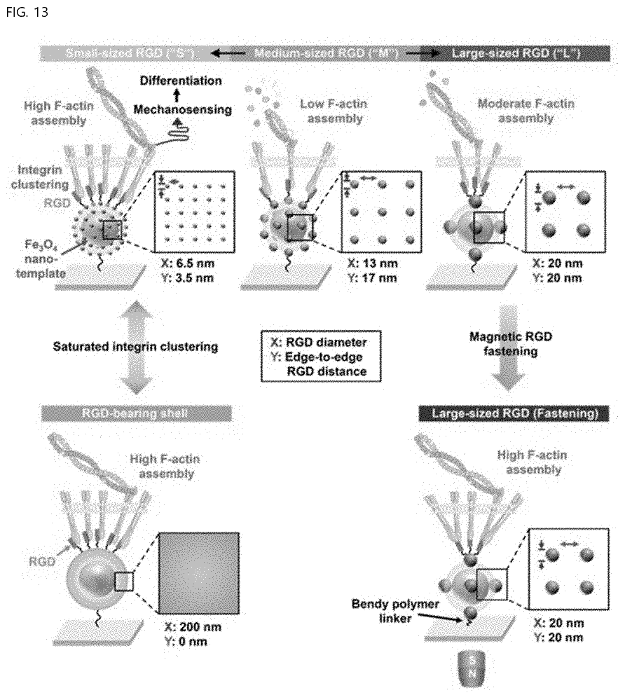

Except for the size of the gold nanoparticles, the magnetic particles, the RGD ligand, the linker, and the substrate were the same as in Experimental Example 2. FIG. 13 is a schematic view showing nanosatellite-substrate complexes prepared by varying the size of gold nanoparticles and the distance between adjacent gold nanoparticles.

1. Preparation of Nanosatellite-Substrate Complexes with Tuned Size of and Distance Between Gold Nanoparticles

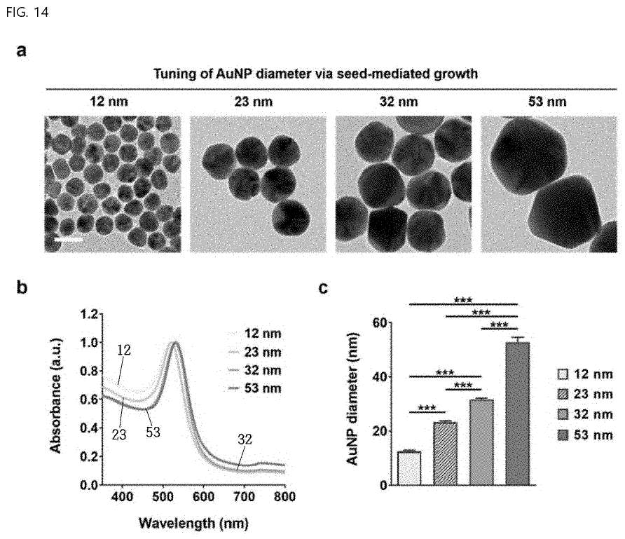

Gold nanoparticles were synthesized by tuning the Au³⁺ concentration, reducing agent, and reaction temperature to tune the size of gold seed particles (3.5 nm and 13 nm). Gold nanoparticles with a diameter of 20 nm were grown from gold seed particles having a diameter of 13 nm. FIG. 14 shows the TEM images, UV-Vis absorbance spectroscopy and diameter of the grown gold nanoparticles. The UV-Vis absorbances of the gold nanoparticles were similar at 520 nm. The diameters of the prepared gold nanoparticles were measured to be 12.4±1.0 nm, 23.2±1.1 nm, 31.6±1.0 nm, and 52.7±4.3 nm. In FIG. 14, the scale bar is 20 nm, and data are expressed as mean±standard error (n=20).

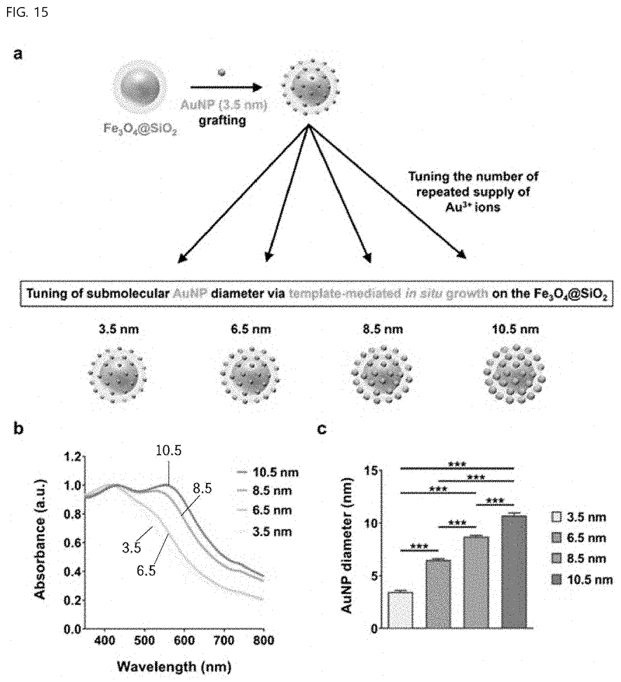

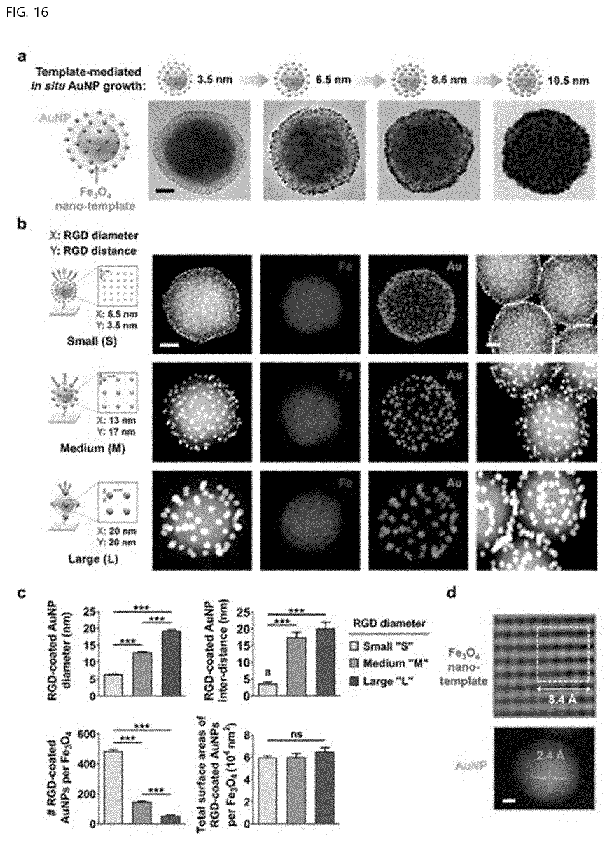

The size of the gold nanoparticles was tuned by supplying Au³⁺ in small amounts and controlling the number of repetitions of supplying Au³⁺ to prevent self-nucleation of gold. FIG. 15 is a schematic diagram showing that the size of gold nanoparticles can be variously tuned adjusted by this method, and the UV-Vis absorbance and diameter of gold nanoparticles prepared by this method. Gold nanoparticles were tuned to 3.5±0.6, 6.5±0.5, 8.7±0.5, and 10.7±1.1 nm on magnetic particles, and as the diameter of the gold nanoparticles increased, the UV-Vis absorption peaks exhibited a red shift. Such changes in the size of the gold nanoparticles can be confirmed by the TEM image in FIG. 16(a) (scale bar: 50 nm).

FIG. 16(b) shows HAADF-STEM and EDS mapping for Small (S), Medium (M), and Large (L) groups depending on the size of gold nanoparticles conjugated to magnetic particles, respectively. It can be confirmed that the size of the gold nanoparticles increases from the S group to the L group, and the distance between adjacent gold nanoparticles increased from the S group to the L group.

FIG. 16(c) shows the results of quantifying the values of S (6.5, 3.5), M (13, 17) and L (20, 20) groups. The diameters of gold nanoparticles in S, M and L groups were 6.4±0.2 nm, 12.9±0.5 nm and 19.3±0.9 nm, respectively, and the distances between adjacent gold nanoparticles in S, M and L groups were 3.6±0.2 nm, 17.4±1.1 nm and 20.1 nm±1.4 nm, respectively, and the numbers of gold nanoparticles per magnetic particle in S, M and L groups were 484.8±10.1 nm, 146.8±4.7 nm and 55.4±2.9 nm, respectively. In addition, the total surface area of gold nanoparticles per magnetic particle was similar between S, M and L groups (59,913 to 64,936 nm²), indicating that the diameter of gold nanoparticles and the distance between adjacent gold nanoparticles were successfully tuned.

In addition, FIG. 16(d) shows the atomic arrangements of crystalline phases of magnetic particles and gold nanoparticles, which were used to confirm the average unit cell lattice parameter and the average lattice spacing, respectively. In FIG. 16(d), the scale bar is 2 nm.

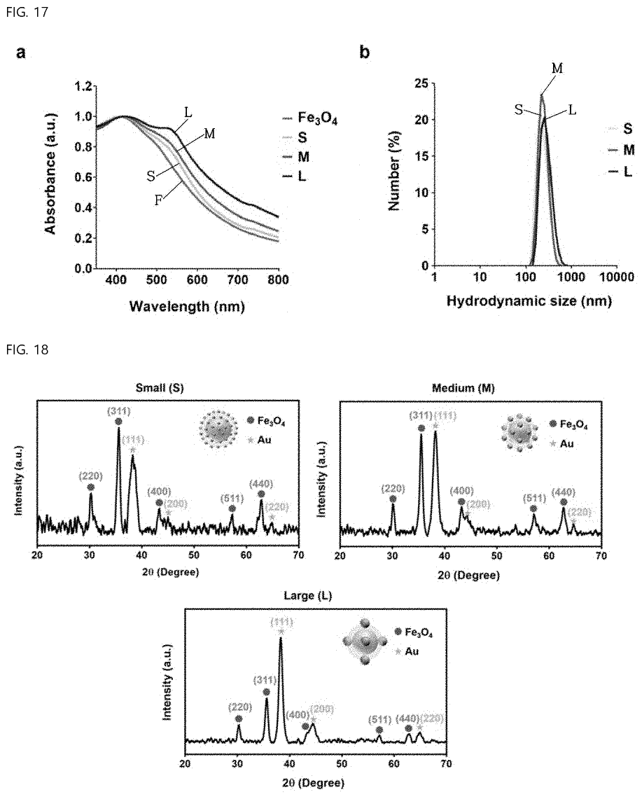

FIG. 17 shows that S, M and L groups all have UV-Vis absorption peaks at 520 nm (gold nanoparticles) and 408 nm (magnetic particles) with similar hydrodynamic diameters.

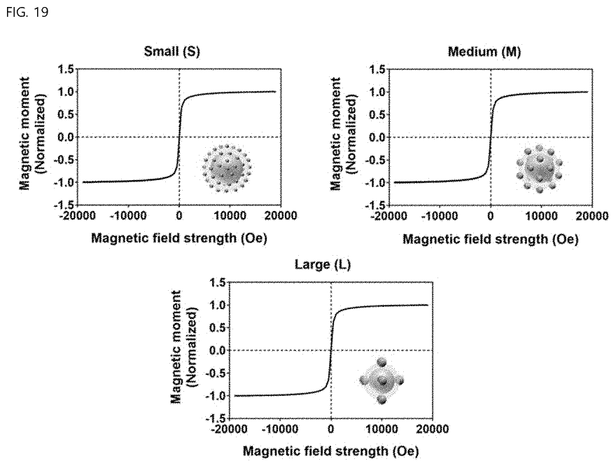

Also, referring to FIG. 18 showing the XRD patterns for each of S, M and L groups and FIG. 19 showing the magnetic reversible properties, all the groups showed similar patterns.

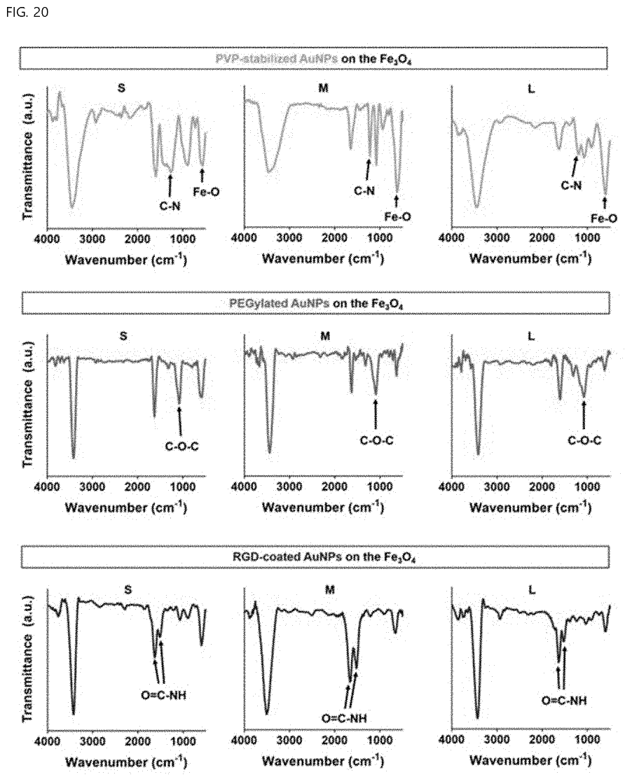

FIG. 20 shows the results of FT-IR in the case where gold nanoparticles were conjugated to magnetic particles and stabilized with PVP for the preparation of each of S, M, and L groups, the case where a PEG linker was conjugated to the gold nanoparticles, and the case where an RGD ligand was coupled to the PEG linker. It can be confirmed that the same functional group exists in each step regardless of the size of the gold nanoparticles.

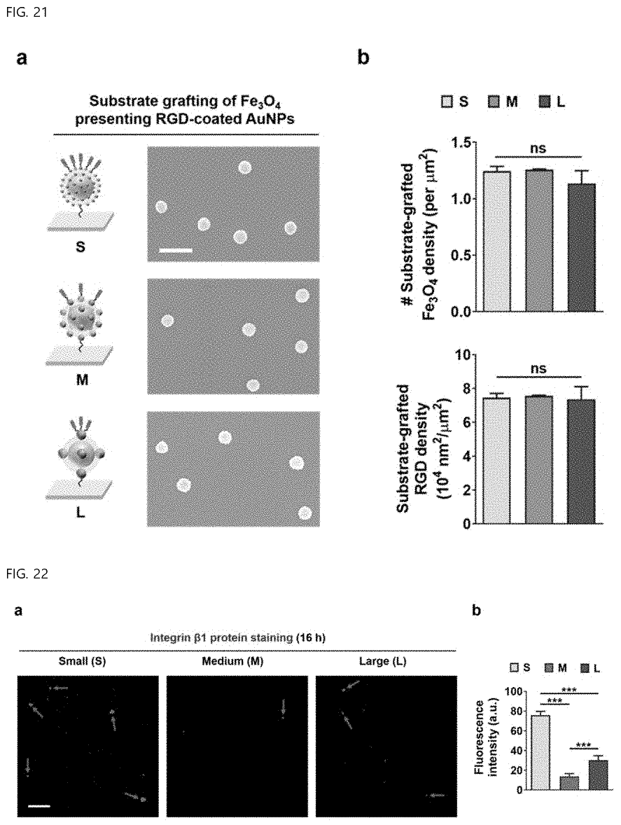

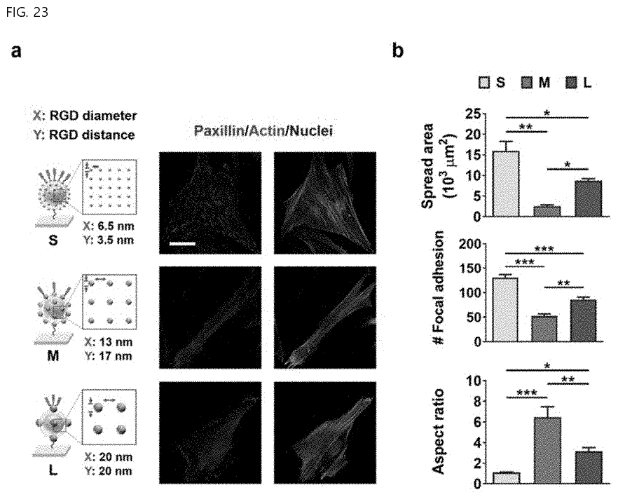

FIG. 21 shows the results of observing the nanosatellite-substrate complex obtained by conjugating the nanoassembly-conjugated magnetic particles to the substrate. In the FE-SEM images (FIG. 21(a)), it can be seen that the magnetic particles were uniformly distributed in each of the S, M, and L groups. In addition, it was confirmed that the distributions of the magnetic particles distributed in all the groups were 1.13 to 1.26 particles/μm², and the global RGD ligand densities of the substrates were similar between the groups (73,648 to 75,613 nm²/μm²).

2. Examination of Integrin Adhesion to Nanosatellite-Substrate Complex

It was tested whether stem cell integrin could bind to the nanosatellite-substrate complex, obtained by tuning the size of and distance between gold nanoparticles, and regulate stem cell differentiation. In order to test whether integrin clustering is facilitated when integrin binds to and bridges the adjacent RGD ligands, the distance between adjacent gold nanoparticles was tuned to a distance (3.5 nm) smaller than the size of the fibronectin (FN) molecule, and the resulting complex was referred to as the S (6.5, 3.5) group. In addition, in order to examine whether integrin clustering is facilitated even when the distance between the RGD ligands is long, the distance between the gold nanoparticles was tuned to a distance (17 nm) larger than the size of the fibronectin (FN) molecule, and the resulting complex was referred to as the M (13. 17) group. In addition, in order to evaluate the case in which integrin binds to each RGD site without forming clusters, the distance between adjacent gold nanoparticles was tuned to a large distance (20 nm), and the resulting complex was referred to as the L (20, 20) group.

FIG. 22 shows confocal microscopic images of nanosatellite-substrate complexes incubated with integrin β1 for 16 hours. It can be confirmed that, in the S group, the fluorescence intensity was strong and abundant, indicating that clustering was facilitated, and it can be confirmed that the fluorescence intensity decreased toward the L group. In addition, the graph (22(b)) quantifying the fluorescence intensity visually shows results. In FIG. 22, the scale bar is 50 μm, and the data are expressed as mean±standard error (n=6).

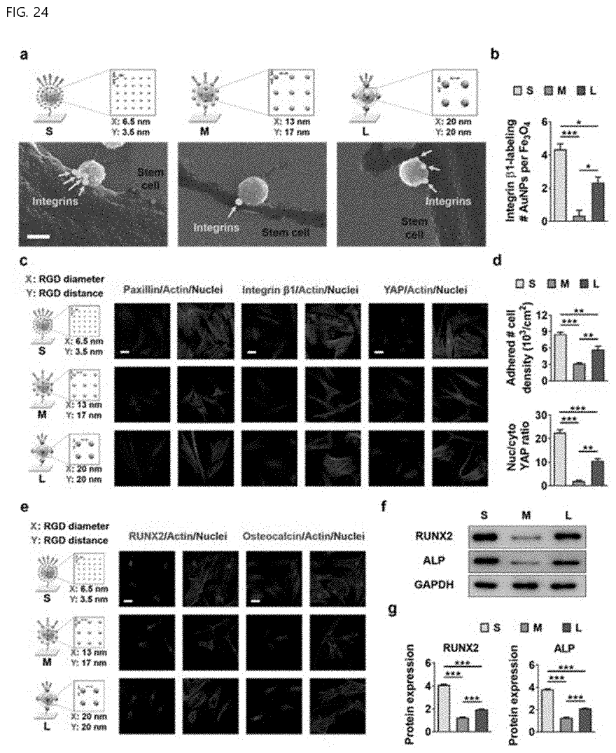

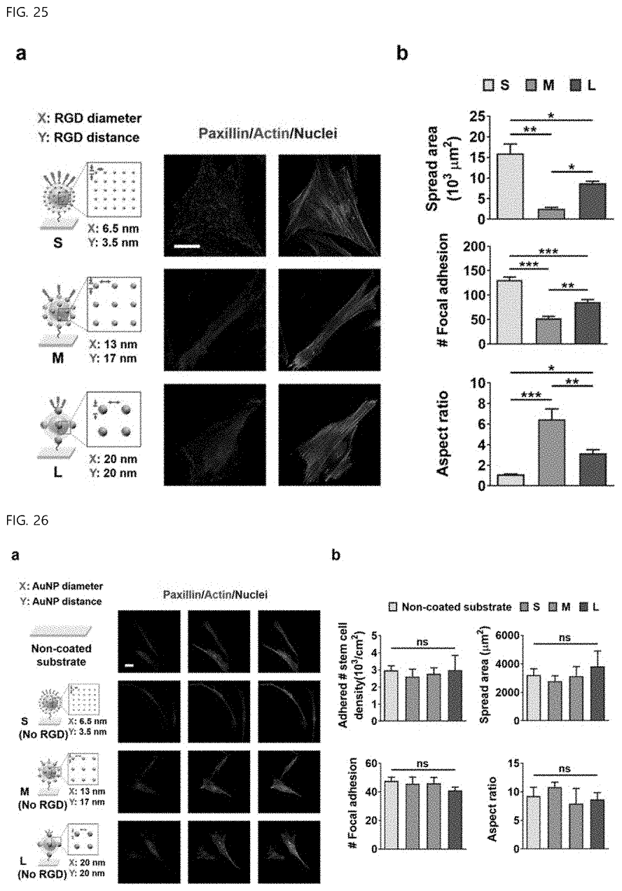

In order to confirm that integrin clustering on each nanosatellite-substrate complex occurs, integrin β1 was labeled with 40-nm gold nanoparticles and then monitored. After the stem cells labeled with the 40-nm gold nanoparticles were cultured for 48 hours, immunofluorescence images of paxillin (focal attachment adapter), F-actin, and YAP (mechanotransducer) showed a considerable increase in stem cell adhesion and the spread of F-actin in the “S” group. However, stem cell adhesion and the spread of F-actin were less obvious in the “M” and “L” groups (FIGS. 24(a) to 24(c) and 25(a)). These results can also be found in the graphs quantifying the number of adhered stem cells, the spread area of the cells, the number of focal adhesions, and the YAP nucleus/cytoplasm ratio (FIGS. 24(d) and 25(b)).

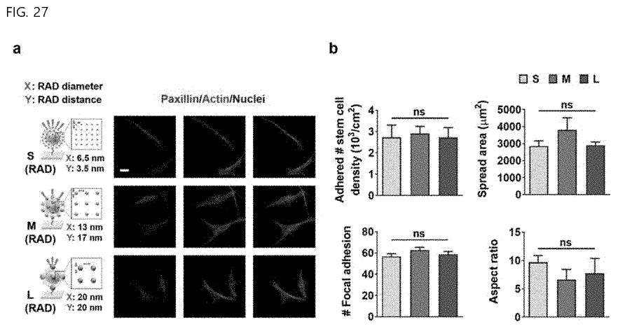

In order to confirm that integrin binds to the RGD ligand, an experiment was conducted by excluding the RGD ligand or the nanosatellite structure from the nanosatellite-substrate complex (FIG. 26) or conjugating the RAD ligand (a scrambled RGD sequence) instead of the RGD ligand (FIG. 27).

In FIG. 26, it can be seen that, even in the case of the S, M and L groups, there was minimal cell adhesion which is similar to that in the case in which only the substrate was present. Therefore, it can be confirmed that the RGD ligand is necessary for stem cell adhesion. In FIGS. 26 and 27, the scale bar is 50 μm.

3. Experiment on Stem Cell Differentiation by Nanosatellite-Substrate Complexes

It was tested whether integrin binds to the nanosatellite-substrate complex to facilitate stem cell differentiation. The nanosatellite-substrate complexes of the S, M and L groups and stem cells were cultured for 4 days in osteogenic induction medium, and then immunofluorescent staining image analysis and Western blotting were performed. Among the early osteogenic markers, RUNX2 represents nuclear translocation, RUNX2 and ALP represent protein expression, and the late osteogenic marker is osteocalcin.

In FIG. 24(e), it can be seen that the expression of all of the markers was remarkably facilitated in the S group, and was relatively inhibited in the M and L groups. In addition, in FIGS. 24(f) and 24(g), it can be seen that the expression of each marker showed the same trend as in FIG. 24(e).

This means that, because the distance between the RGD ligands in the S group is short, integrin clustering and mechanotransduction signaling are promoted.

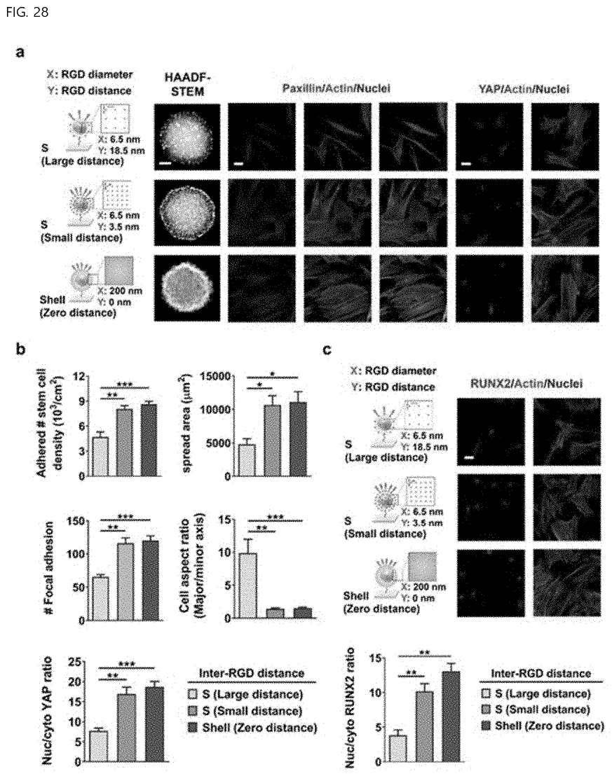

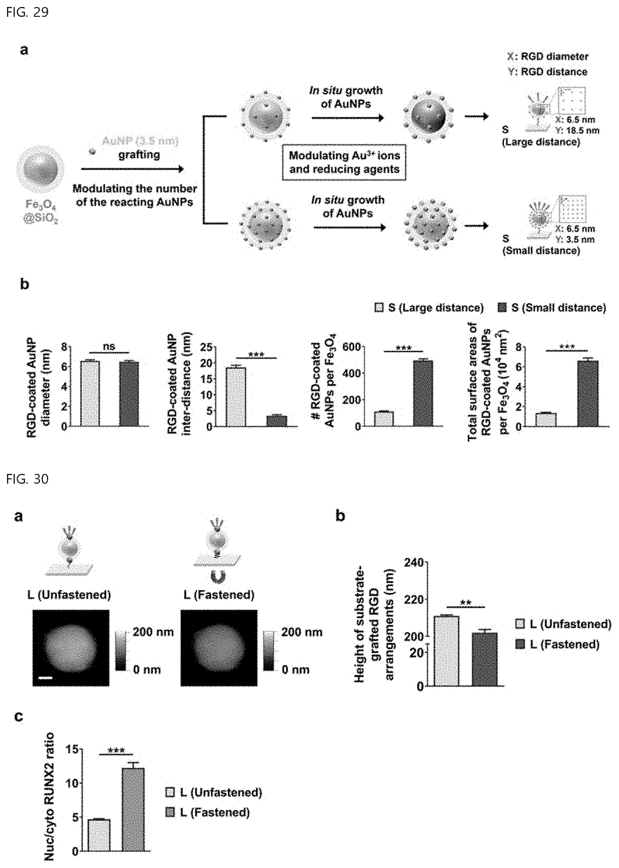

FIGS. 28 and 29 show the results of examining the degree of stem cell adhesion and differentiation by maintaining the size of the gold nanoparticles at 6.5 nm and tuning only the distance between gold nanoparticles. From the immunofluorescence image and the graph quantifying the same, it can be seen that as the distance between the gold nanoparticles was shorter, stem cell adhesion and differentiation was more facilitated. However, when comparing the case where the distance between the adjacent gold nanoparticles was 3.5 nm and the case where the entire surface of the magnetic particle was coated with the nanoassembly, it can be seen that there is no significant difference in the degree of stem cell adhesion and differentiation between these cases.

Therefore, it can be seen that, when the distance between adjacent gold nanoparticles is less than 3.5 nm, the integrin recognizes and binds to the RGD ligand. In other words, it can be seen that, when the distance between gold nanoparticles is 3.5 nm, it is the largest distance among the distances that can induce saturated integrin clustering.

In addition, when the distance between gold nanoparticles is increased to 17 nm or more, clustering of integrins bound to the neighboring RGD ligands is difficult to induce, and thus stem cell adhesion and differentiation is inhibited. This distance is a distance exceeding 16 nm, which is the size of fibronectin (FN). When this distance is exceeded, even if the size of the gold nanoparticles is increased, integrin clustering is not induced, and thus stem cell adhesion and differentiation is inhibited.

However, when the diameter of the gold nanoparticles exceeds 20 nm, even if the distance between the adjacent gold nanoparticles is larger than the size of fibronectin (for example, 20 nm), integrin clustering may be induced on one gold nanoparticles, and stem cells adhesion and differentiation may be facilitated.

This is because a plurality of integrin molecules can bind to a gold nanoparticle, and these integrin molecules have the property of approaching each other in order to dissipate energy from the outside.

This trend is the same as in Experimental Example 2 in which only the distance between the gold nanoparticles was tuned.

### Experimental Example 4

It was tested whether stem cell adhesion and differentiation could be regulated by applying a magnetic field to the nanosatellite-substrate complex to move the nanosatellite structure.

1. Effect Depending on Direction of Movement of Nanosatellite-Substrate Complex

It was tested whether stem cells could be regulated by applying a magnetic field to the nanosatellite-substrate complex to move the nanosatellite structure.

In order to examine whether the degree of stem cell adhesion and differentiation varies depending on the direction in which the nanosatellite structure is moved by applying a magnetic field, an experiment was conducted by applying a magnetic field to the nanosatellite-substrate complex L (20, 20) in which the size of the gold nanoparticles and the distance between the adjacent gold nanoparticles are the same.

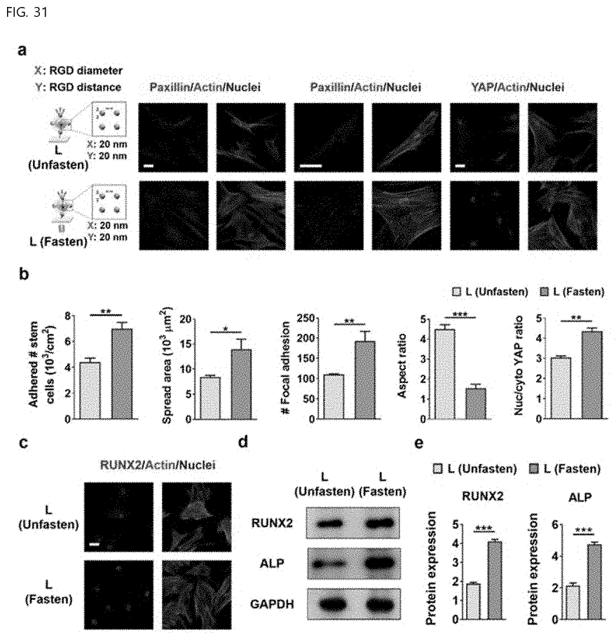

In FIGS. 30 and 31, L (Unfastened) is a state in which no magnetic field was applied, and L (Fastened) is a case in which the nanosatellite structure was moved in a direction toward the substrate by applying a magnetic field to the lower side of the substrate.

In FIG. 30, it can be seen that the shading of the magnetic particles is different between L (unfastened) and L (fastened), indicating that the nanosatellite structure was vertically moved by the application of a magnetic field. Even in the graphs showing the height of the magnetic particles by quantification, it can be seen that the height in the L (fastened) group (darker contrast, 221.0±1.6 nm) was different from that in the L (unfastened) group.

In addition, immunofluorescence-stained images, western blotting, and corresponding quantification analysis of the adhered number of stem cells, spread cell area, number of focal adhesions, nucleus/cytoplasm YAP and RUNX2 ratio, and RUNX2 and ALP expression consistently revealed a higher degree of stem cell adhesion and marker expression in the L (Fastened) group than in the L (Unfastened) group.

2. Effect Depending on Size and Density of Gold Nanoparticles (Nanoassemblies)

It was tested whether stem cell adhesion and differentiation could be regulated by tuning the size of gold nanoparticles (nanoassemblies) and the distance between adjacent gold nanoparticles and applying a magnetic field thereto.

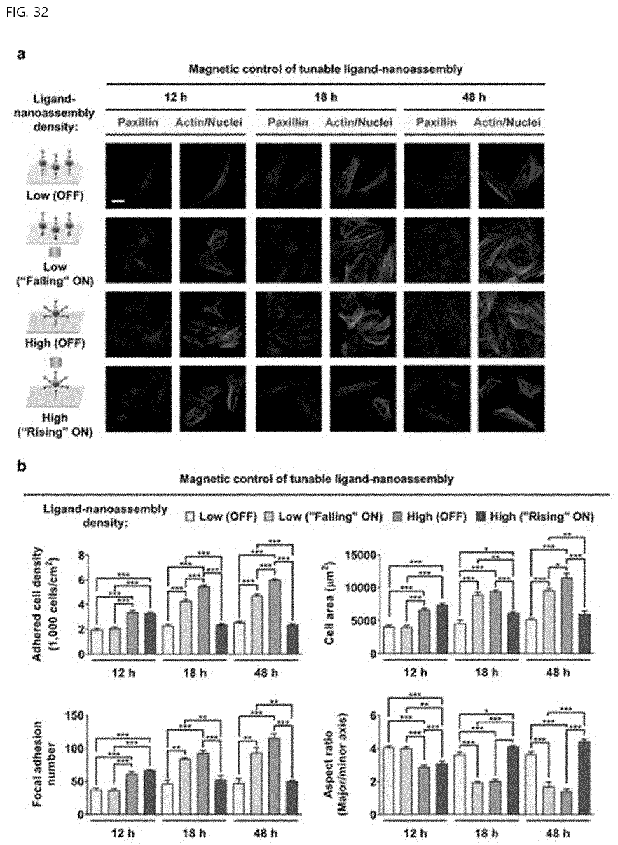

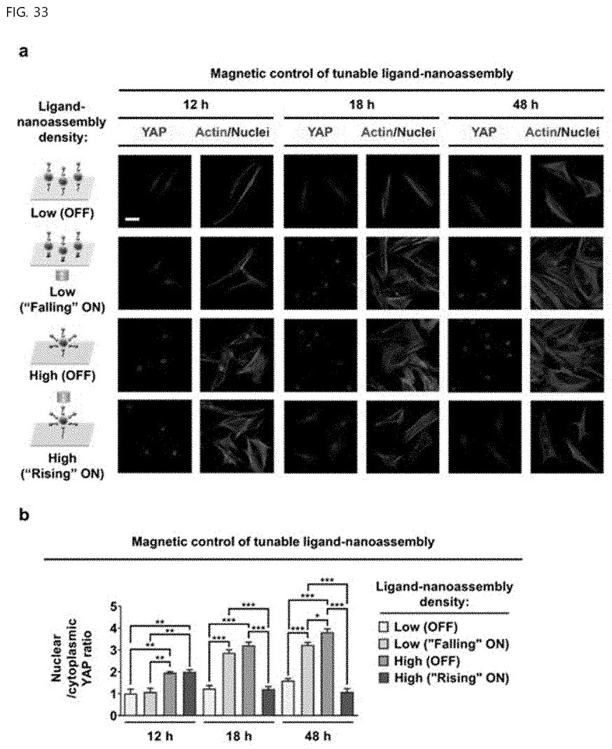

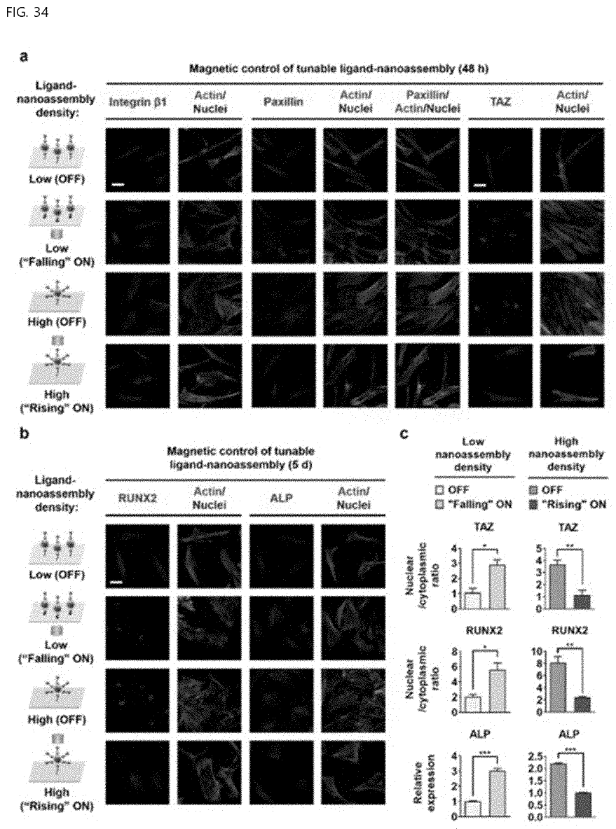

FIGS. 32 to 34 show the results of comparing the degree of stem cell adhesion and marker expression by varying (Low or High) the density of the nanoassembly conjugated to the magnetic particle and applying a magnetic field thereto. In FIGS. 32 to 34, the scale bar is 50 μm.

It can be confirmed that stem cell adhesion and differentiation was inhibited when no magnetic field was applied (“OFF” state), but was facilitated at “Low”. However, when the “Falling” ON state was created by applying a magnetic field at “Low”, stem cell adhesion and differentiation was facilitated to a level similar to that in OFF at High. In addition, when a magnetic field was applied at High to create the “Rising” ON state, stem cell adhesion and differentiation was inhibited to a degree similar to that in OFF at Low. In addition, it can be confirmed that stem cell adhesion and differentiation was regulated even after 18 hours after the application of the magnetic field.

These results can also be found in the graphs quantifying the density of adhered stem cells, cell area, number of focal adhesion, aspect ratio, and nucleus/cytoplasm YAP ratio.

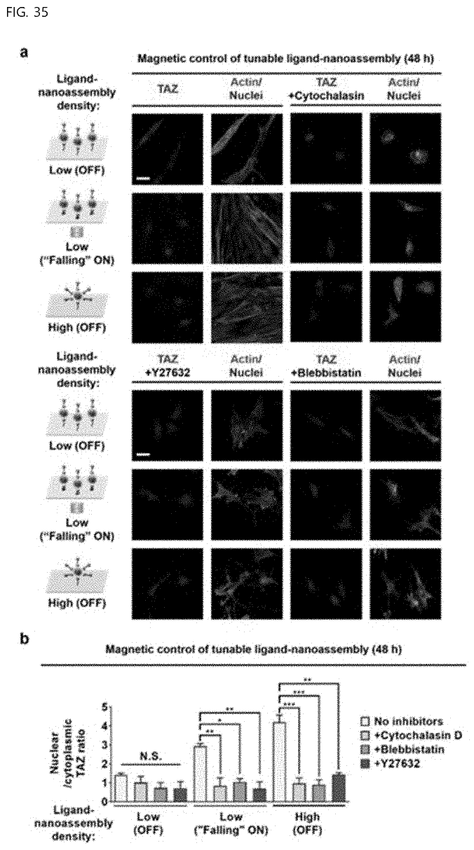

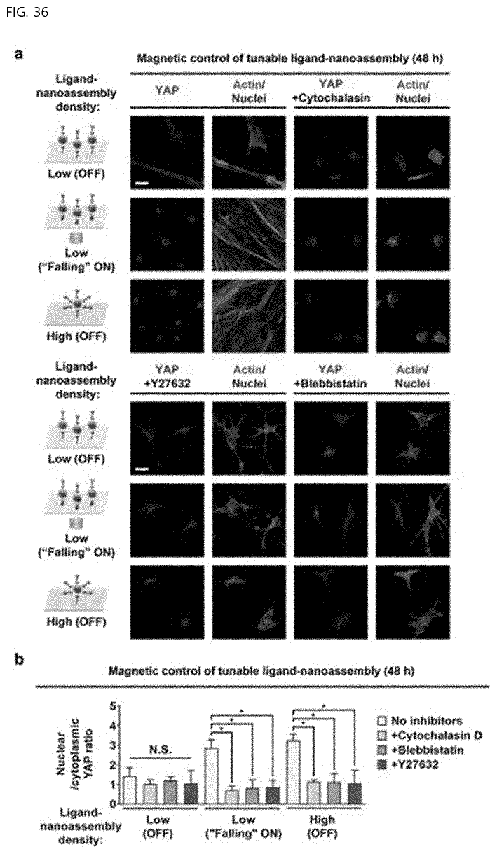

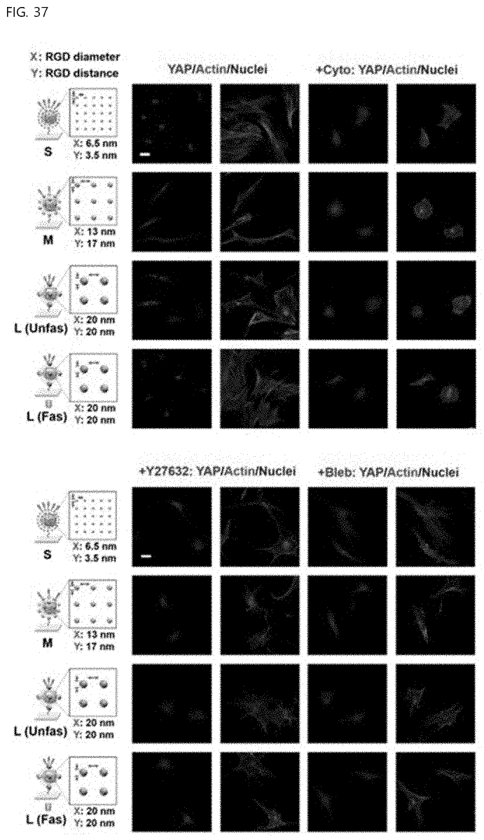

FIGS. 35 and 36 show the results of an experiment conducted culturing for 48 hours in the presence of an inhibitor specific to each protein. It can be seen that the expression of YAP, TAZ and actin/nucleus significantly decreased in the presence of the inhibitor compared to in the absence of the inhibitor, suggesting that stem cell adhesion decreased in the presence of the inhibitor.

This trend was the same even when both the diameter of the gold nanoparticles and the distance between the adjacent gold nanoparticles were tuned.

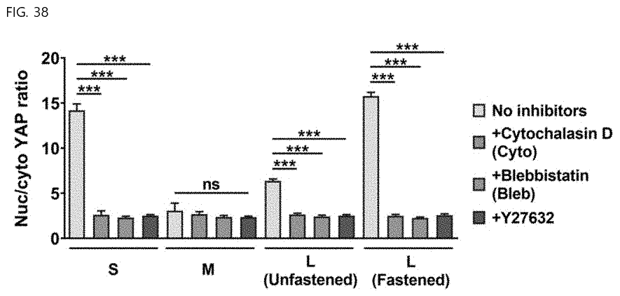

FIGS. 37 and 38 show the results of an experiment conducted on stem cell adhesion and differentiation by dividing gold nanoparticles into S, M and L groups based on the diameter of the gold nanoparticles and the spacing between adjacent gold nanoparticles, and applying a magnetic field to the L group. It can be seen that, when no magnetic field was applied, stem cell adhesion was facilitated is promoted in the neighboring S group, and decreases toward the L group. However, it can be confirmed that, when a magnetic field was applied to the L group to place the nanoassembly in the Fas state, stem cell adhesion was facilitated to a degree similar degree to that in the S group.

In addition, it can be confirmed that, when cytochalasin D, Y27632 and blebbistatin, which are proteins that inhibit actin polymerization, rho-associated protein kinase (ROCK) and myosin II, respectively, were added, stem cell adhesion and nucleus/cytoplasm YAP ratio significantly decreased, suggesting that these molecules were involved in mechanotransduction.

These experimental results show that, when the nanoassembly density is low and when the diameter of the gold nanoparticles is large and the distance between the adjacent gold nanoparticles is large, stem cell adhesion and differentiation is inhibited, but when a magnetic field is applied thereto to create the “falling” ON state or the Fastened state, stem cell adhesion and differentiation may be facilitated. On the other hand, these results show that, when the nanoassembly density is high and when the diameter of gold nanoparticles is small and the distance between adjacent gold nanoparticles is small, stem cell adhesion and differentiation is facilitated, and when a magnetic field is applied thereto to create the “Rising” ON state or the Unfastened state, stem cell adhesion and differentiation may be inhibited.

These results mean that it is possible to repair damaged tissues based on stem cells by regulating stem cell adhesion and differentiation.

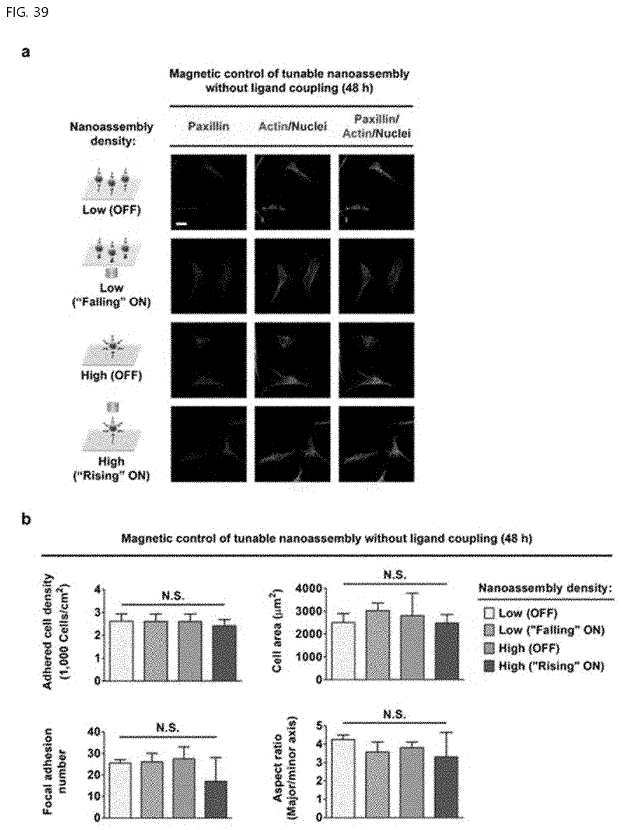

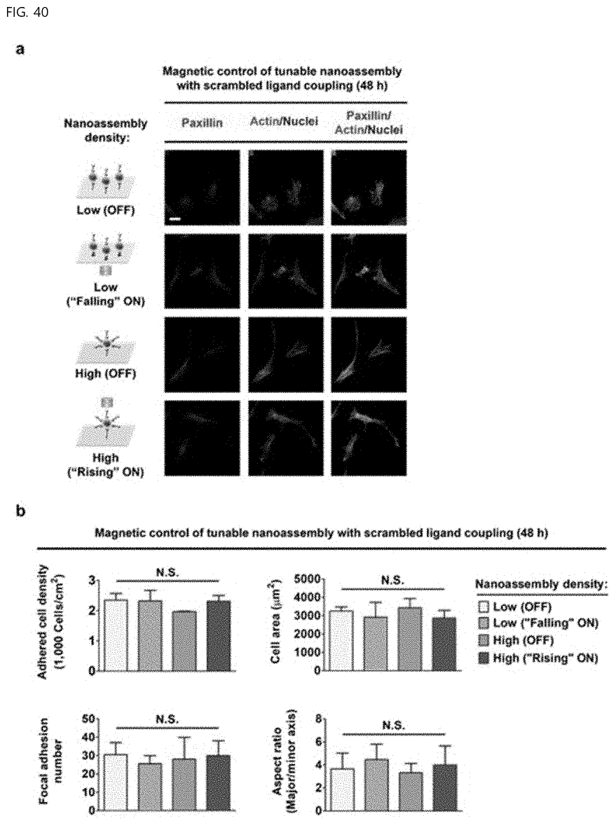

As a result of conducting the same experiment on the nanosatellite-substrate complex that does not contain the ligand and the nanosatellite-substrate complex containing the RAD ligand instead of the RGD ligand, it could be confirmed again that stem cell adhesion and differentiation hardly occurred, indicating that the RGD ligand is necessary for regulating stem cell adhesion and differentiation (FIGS. 39 and 40).

### Experimental Example 5

In order to examine whether the above-described experimental results work even in an actual living body and examine the in vivo stability of the nanosatellite-substrate complex, an experiment was conducted by subcutaneously implanting the nanosatellite-substrate complex into mice and injecting hMSCs into the mice.

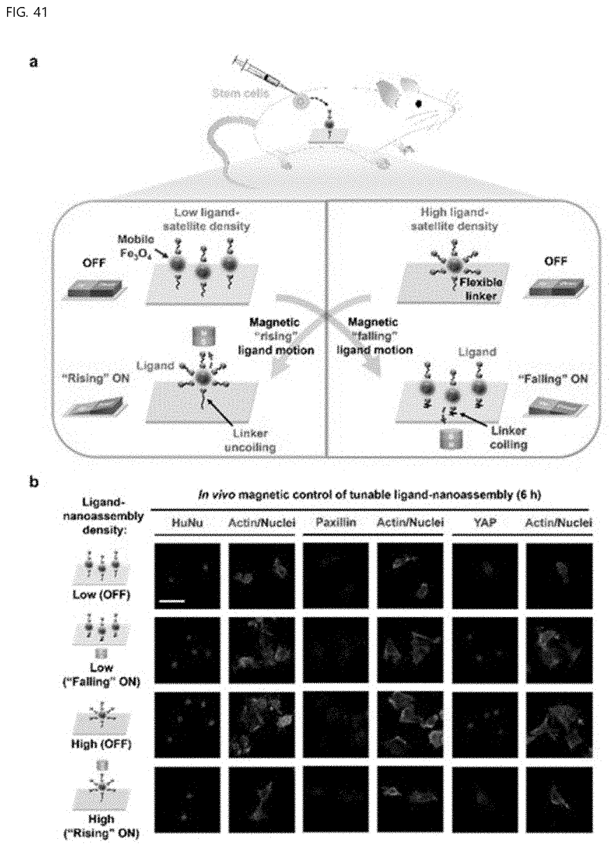

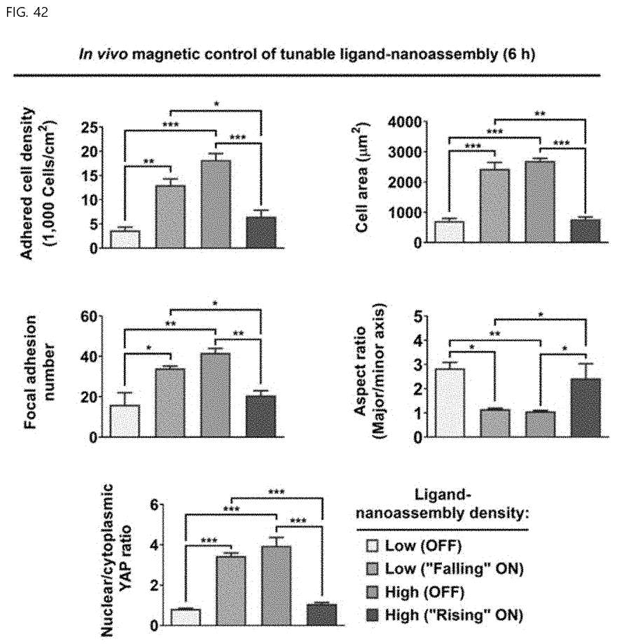

FIGS. 41 and 42 show the results of experiments conducted after nanosatellite-substrate composites, prepared to have different nanoassembly densities, into mice. It can be confirmed that, even when the composite was implanted in vivo, as in Experimental Example 4, in the case in which no magnetic field was applied, stem cell adhesion and differentiation was inhibited when the nanoassembly density was low, but stem cell adhesion and differentiation was facilitated when the nanoassembly density was high. In addition, it can be confirmed that, when a magnetic field was applied to create the “Falling” ON state, stem cell adhesion and differentiation was facilitated, and when the “Rising” ON state was created, stem cell adhesion and differentiation was inhibited.

These results can also be found in FIG. 42 which show the results of quantifying the adhered cell density, cell area, number of focal adhesions, aspect ratio, and nucleus/cytoplasm YAP ratio.

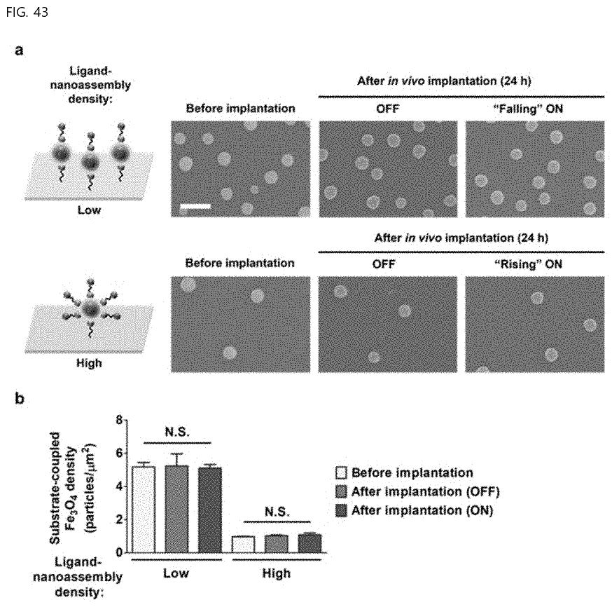

FIG. 43 shows the results of evaluating the in vivo stability of the nanosatellite-substrate complex. It can be confirmed that there was little change in the density, shape and position of the nanosatellite-substrate complex between before and after implantation of the composite into the mice and even after application of the magnetic field, indicating that the nanosatellite-substrate complex was stable in vivo.

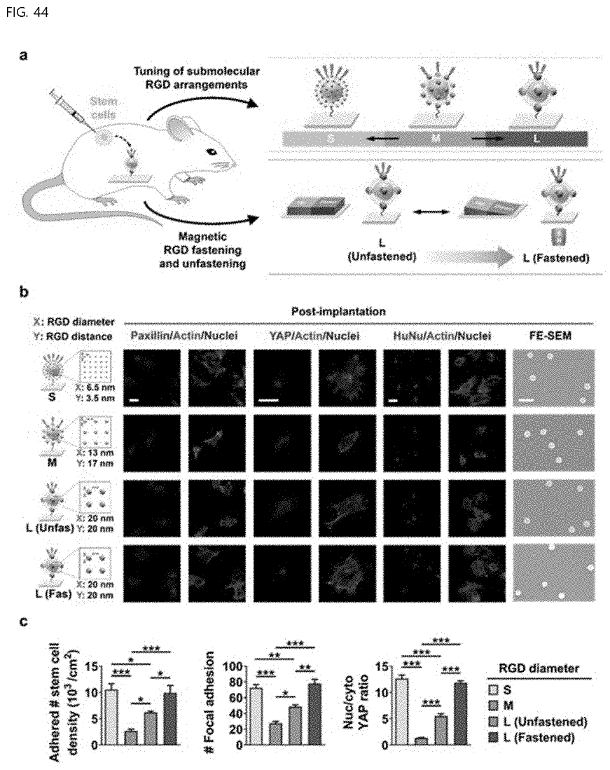

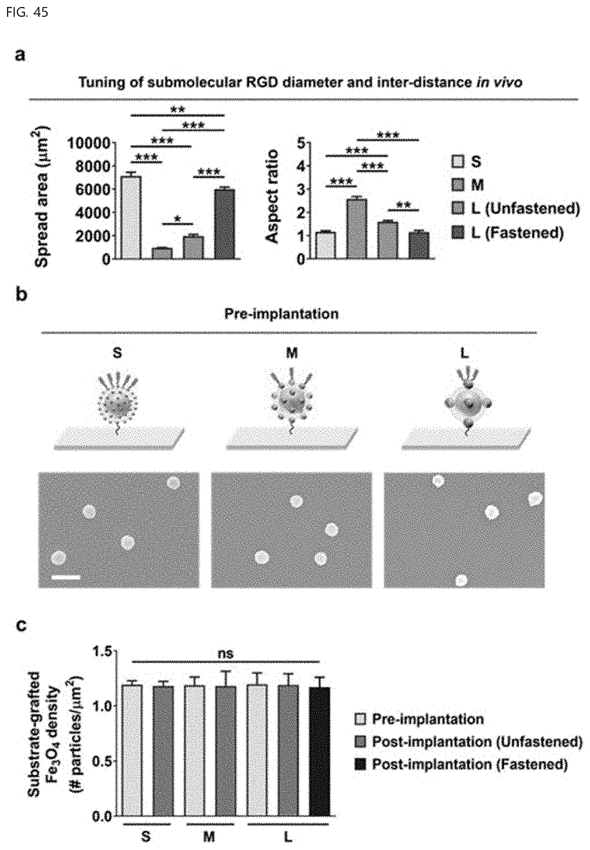

FIGS. 44 and 45 show the results of an experiment conducted by in vivo implantation of nanosatellite-substrate complexes prepared by tuning the diameter of gold nanoparticles and the distance between adjacent gold nanoparticles.

It can be confirmed that, the S group in which the gold nanoparticles have a small diameter and the distance between adjacent gold nanoparticles is small, stem cell adhesion was facilitated, whereas stem cell adhesion was inhibited toward the L group. On the other hand, it can be confirmed that, when a magnetic field was applied to the L group to create the Fas state, stem cell adhesion was facilitated to a degree similar to that in the S group.

Also in this case, it can be confirmed that the nanosatellite-substrate complex implanted into the mice was not degraded or modified, indicating that it was stable in vivo.

These results mean that regulation of stem cell adhesion and differentiation using the nanosatellite-substrate complex in vitro is possible even in vivo.

As described above, according to the present invention, it is possible to provide a nanosatellite-substrate complex capable of regulating stem cell adhesion and differentiation.

In addition, according to the present invention, it is possible to provide a method for preparing a nanosatellite-substrate complex capable of regulating stem cell adhesion and differentiation.

In addition, according to the present invention, it is possible to provide a method of regulating stem cell adhesion and differentiation using the nanosatellite-substrate complex.

While the present invention has been described with reference to the particular illustrative embodiments, it will be understood by those skilled in the art to which the present invention pertains that the present invention may be embodied in other specific forms without departing from the technical spirit or essential characteristics of the present invention. Therefore, the embodiments described above are considered to be illustrative in all respects and not restrictive. Furthermore, the scope of the present invention is defined by the appended claims rather than the detailed description, and it should be understood that all modifications or variations derived from the meanings and scope of the present invention and equivalents thereto are included in the scope of the present invention.

What is claimed is:

1. A nanosatellite-substrate complex for regulating stem cell adhesion and differentiation comprising:
a substrate;
a core-shell-type magnetic particle provided to be spaced apart from at least one side of the substrate;
a gold nanoparticle connected to one side of the magnetic particle;
a first linker connecting between the substrate and the gold nanoparticle;
a second linker connecting between the gold nanoparticle and a ligand; and
the ligand connected to the gold nanoparticles via the second linker,
wherein the gold nanoparticle is connected to the ligand via the second linker to form a nanoassembly,
the magnetic particle is conjugated to the nanoassembly to form a nanosatellite structure,
the nanoassembly comprises one or more gold nanoparticles, one or more second linkers connected to at least one of the one or more gold nanoparticles, and the ligand connected to the second linker, and
one or more nanoassemblies are comprised in the nanosatellite structure.

2. The nanosatellite-substrate complex of claim 1, wherein the magnetic particle is composed of:
a core composed of iron oxide; and
a shell provided to cover an outer surface of the core and comprising silica.

3. The nanosatellite-substrate complex of claim 1, wherein the gold nanoparticles have at least one diameter selected from among a first average diameter, a second average diameter, and a third average diameter,
wherein the first average diameter is 3.5 nm to 10.5 nm, the second average diameter is 12 nm to 14 nm, and the third average diameter is 15 nm to 25 nm.

4. The nanosatellite-substrate complex of claim 1, wherein a plurality of the nanoassemblies are provided adjacent to each other, and a distance between the gold nanoparticles in each of the nanoassemblies provided adjacent to each other includes at least one of a first distance, a second distance and a third distance,
wherein the first distance is 3 nm to 4 nm, the second distance is 15 nm to 20 nm, and the third distance is 18 nm to 22 nm.

5. The nanosatellite-substrate complex of claim 1, wherein the nanoassemblies are provided to completely cover an outer surface of the magnetic particle.

6. The nanosatellite-substrate complex of claim 1, wherein the magnetic particle has an average diameter of 150 nm to 250 nm, and comprises at least one of an amino group (—HN₂) and a thiol group (—SH) on a surface thereof.

7. The nanosatellite-substrate complex of claim 1, wherein the first linker and the second linker have a structure of the following Formula 1: 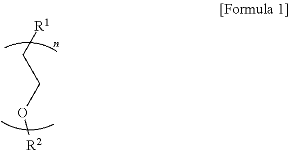
wherein R¹ is one of a thiol group (—SH) and an amine group (—NH₂), R² is one of a carboxyl group (—COOH), an amine group (—NH₂) and a succinimidyl ester group, and n is a number ranging from 113 to 450.

8. The nanosatellite-substrate complex of claim 1, wherein the ligand is a cyclic RGD ligand.

9. The nanosatellite-substrate complex of claim 1, wherein a surface of the nanosatellite structure, which faces the substrate, is spaced apart from the substrate with the first linker interposed therebetween, and the first linker is elastic and a length thereof is reversibly changed by application of a magnetic field.

10. The nanosatellite-substrate complex of claim 1, wherein
the nanosatellite structure is provided to be spaced apart from one side of the substrate,
the first linker is compressed by applying a magnetic field to the other side of the substrate, and
the nanosatellite structure moves in a direction toward the substrate to facilitate stem cell adhesion and differentiation.

11. The nanosatellite-substrate complex of claim 1, wherein
the nanosatellite structure is provided to be spaced apart from one side of the substrate,
the first linker is stretched by applying a magnetic field to an upper side of the nanosatellite structure, which is one side of the substrate, and
the nanosatellite structure moves in a direction away from the substrate to inhibit stem cell adhesion and differentiation.

12. The nanosatellite-substrate complex of claim 1, wherein a density of the nanosatellite structure provided on the substrate is 1.0 nanosatellite structure/μm² to 6 nanosatellite structures/μm².

13. A method for preparing a nanosatellite-substrate complex for regulating stem cell adhesion and differentiation, the method comprising:
coating a surface of iron oxide with a silica having an amine group to form magnetic particles;
providing gold nanoparticles on surfaces of the magnetic particles;
adding and dispersing the magnetic particles having the gold nanoparticles provided thereon in a solution containing a polymer linker to form a first linker and a second linker;
reacting the first linker with a substrate having amine groups formed thereon, so that the first linker is bound to at least a portion of the amine groups formed on the substrate and the magnetic particles having the gold nanoparticles provided thereon are conjugated to the substrate;
deactivating amine groups, which remain unbound to the first linker on the substrate, by treatment with a deactivating group; and
conjugating a ligand to the second linker.

14. The method of claim 13, wherein the gold nanoparticles have any one diameter selected from among a first average diameter, a second average diameter and a third average diameter;
the first average diameter of the gold nanoparticles is 3.5 nm to 10.5 nm, and
the gold nanoparticles having the first average diameter are formed by reacting first gold seed particles with amine groups on the surfaces of the magnetic particles to provide the gold seed particles on the magnetic particles, and adding and stirring the magnetic particles having the gold seed particles provided thereon in a gold-containing solution to grow the gold seed particles; or
the second average diameter of the gold nanoparticles is 12 nm to 14 nm and the third average diameter thereof is 15 nm to 25 nm, and
the gold nanoparticles having the second or third average diameter are provided on the magnetic particles by adding and stirring second gold seed particles in a gold-containing solution to grow the second gold seed particles, thereby forming gold nanoparticles, and reacting the gold nanoparticles, formed by growing the second gold seed particles, with amine groups on the surfaces of the magnetic particles.

15. The method of claim 14, wherein the gold-containing solution comprises a first solution containing sodium citrate and a second solution containing chloroauric acid,
the first solution and the second solution are sequentially added, and
the average diameter of the gold nanoparticles is modulated by controlling the number of times the first solution and the second solution are added.

16. A method of regulating stem cell adhesion and differentiation using a nanosatellite-substrate complex, the method comprising regulating stem cell adhesion and differentiation by applying a magnetic field to the nanosatellite-substrate complex according to claim 1.

17. The method of claim 16, wherein the magnetic field is applied from outside the body to remotely control the nanosatellite-substrate complex in the body.

18. The method of claim 16, wherein the magnetic field has a strength of 100 mT to 500 mT.

19. The method of claim 16, wherein the nanosatellite structure is provided to be spaced apart from one side of the substrate,
a plurality of the nanoassemblies are provided adjacent to each other in the nanosatellite structure,
the gold nanoparticles have an average diameter of 3.5 nm to 10.5 nm,
a distance between the gold nanoparticles in the nanoassemblies provided adjacent to each other is 3 nm to 4 nm,
the first linker is stretched by applying a magnetic field to an upper side of the nanosatellite structure, which is one side of the substrate, and
the nanosatellite structure moves in a direction away from the substrate to inhibit stem cell adhesion and differentiation.

20. The method of claim 16, wherein the nanosatellite structure is provided to be spaced apart from one side of the substrate,
a plurality of the nanoassemblies are provided adjacent to each other in the nanosatellite structure,
the gold nanoparticles have an average diameter of 15 nm to 25 nm,
a distance between the gold nanoparticles in the nanoassemblies provided adjacent to each other is 18 nm to 22 nm,
the first linker is compressed by applying a magnetic field to the other side of the substrate, and
the nanosatellite structure moves in a direction toward the substrate to facilitate stem cell adhesion and differentiation.
